(19) United States
(12) Patent Application Publication
(10) Pub. No.: US 2011/0060012 A1
(43) Pub. Date: Mar. 10, 2011

(54) 4-[3-(ARYLOXY)BENZYLIDENE]-3-METHYL PIPERIDINE 5-MEMBERED ARYL CARBOXAMIDE COMPOUNDS USEFUL AS FAAH INHIBITORS

(75) Inventors: Marvin Jay Meyers, Wentzville, MO (US); Matthew James Pelc, Kenosha, WI (US); Atli Thorarensen, Stow, MA (US); Barbara Ann Schweitzer, St. Louis, MO (US)

(73) Assignee: Pfizer Inc.

(21) Appl. No.: 12/936,946

(22) PCT Filed: Apr. 9, 2009
(86) PCT No.: PCT/IB09/05254

§ 371 Date: Nov. 18, 2010

Related U.S. Application Data
(60) Provisional application No. 61/045,881, filed on Apr. 17, 2008.

(51) Int. Cl. A61K 31/4545; C07D 401/14; A61P 9/00; A61P 25/00; A61P 29/00; A61P 27/06; A61P 17/06; A61P 1/00

(52) U.S. Cl. 514/340; 546/194

(57) ABSTRACT

The present invention relates to compounds of Formula (I), wherein Ar is a 5-membered heteroaryl moiety and X, Y and Z are independently N or CH; or a pharmaceutically acceptable salt thereof; processes for the preparation of the compounds; intermediates used in the preparation of the compounds; compositions containing the compounds; and uses of the compounds in treating diseases or conditions associated with fatty acid amide hydrolase (FAAH) activity.

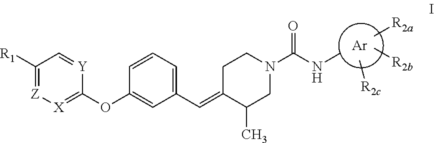

### FIELD OF THE INVENTION

The present invention relates to 4-[3-(aryloxy)benzylidene]-3-methyl piperidine aryl carboxamide compounds and the pharmaceutically acceptable salts of such compounds. The invention also relates to the processes for the preparation of the compounds, intermediates used in their preparation, compositions containing the compounds, and the uses of the compounds in treating diseases or conditions associated with fatty acid amide hydrolase (FAAH) activity.

### BACKGROUND OF THE INVENTION

Fatty acid amides represent a family of bioactive lipids with diverse cellular and physiological effects. Fatty acid amides are hydrolyzed to their corresponding fatty acids by an enzyme known as fatty acid amide hydrolase (FAAH). FAAH is a mammalian integral membrane serine hydrolase responsible for the hydrolysis of a number of primary and secondary fatty acid amides, including the neuromodulatory compounds anandamide and oleamide. Anandamide (arachidonoyl ethanolamide) has been shown to possess cannabinoid-like analgesic properties and is released by stimulated neurons. The effects and endogenous levels of anandamide increase with pain stimulation, implying its role in suppressing pain neurotransmission and behavioral analgesia. Supporting this, FAAH inhibitors that elevate brain anandamide levels have demonstrated efficacy in animal models of pain, inflammation, anxiety, and depression. Lichtman, A. H. et al. (2004), J. Pharmacol. Exp. Ther. 311, 441-448; Jayamanne, A. et al. (2006), Br. J. Pharmacol. 147, 281-288; Kathuria, S. et al. (2003), Nature Med., 9, 76-81; Piomelli D. et al (2005), Proc. Natl. Acad. Sci . . . 102, 18620-18625.

PCT Application No. PCT/IB2007/003202, filed Oct. 5, 2007, and published as WO2008/047229 on 24 Apr. 2009, concerns biaryl ether compounds that are inhibitors of FAAH. PCT Application WO 2006/085196 teaches a method for measuring activity of an ammonia-generating enzyme, such as FAAH WO 2006/067613 relates to compositions and methods for expression and purification of FAAH. WO2006/074025 concerns piperazinyl and piperidinyl ureas as FAAH modulators.

There remains a need for new compounds that are inhibitors of FAAH and, therefore, are useful in the treatment of a wide range of disorders, including pain.

### SUMMARY OF THE INVENTION

The present invention relates to compounds of the Formula I:

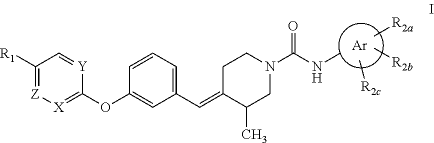

wherein:

Ar is a 5-membered heteroaryl moiety;

X, Y and Z are independently selected from CH or N;

R₁ is H, C₁-C₆ alkyl, C₁-C₆ alkoxy, halogen, C₁-C₃ haloalkyl, C₁-C₃ haloalkoxy, C₃-C₆ cycloalkyl, —CH₂—C₃-C₆ cycloalkyl, C₃-C₆ cycloalkoxy, —CH₂—C₃-C₆ cycloalkoxy, or CN; with the rings of the C₃-C₆ cycloalkyl, —CH₂—C₃-C₆ cycloalkyl, C₃-C₆ cycloalkoxy, and —CH₂—C₃-C₆ cycloalkoxy groups being optionally substituted by from 1 to 4 fluorine atoms;

R₂ₐ is H, C₁-C₆ alkyl, C₁-C₆ alkoxy, C₂-C₆ alkenyl, C₂-C₆ alkynyl, halogen, C₁-C₃ haloalkyl, C₁-C₃ haloalkoxy, C₃-C₈ cycloalkyl, —(CH₂)ₙ—C₃-C₈ cycloalkyl, C₃-C₈ cycloalkoxy, C₅-C₈ cycloalkenyl, —(CH₂)ₙ—C₅-C₈ cycloalkenyl, C₅-C₈ cycloalkenyloxy, 4- to 8-membered heterocycle containing from 1 to 3 ring heteroatoms selected from O, S and N, —(CH₂)ₙ-(4- to 8-membered heterocycle containing from 1 to 3 ring heteroatoms selected from O, S and N), —(CH₂)ₙ—O-(4- to 8-membered heterocycle containing from 1 to 3 ring heteroatoms selected from O, S and N) or CN; with:
a) the R₂ₐ C₃-C₈ cycloalkyl, —(CH₂)ₙ—C₃-C₈ cycloalkyl, C₃-C₈ cycloalkoxy, C₅-C₈ cycloalkenyl, (CH₂)ₙ—C₅-C₈ cycloalkenyl, C₅-C₈ cycloalkenyloxy, 4- to 8-membered heterocycle containing from 1 to 3 ring heteroatoms selected from O, S and N, —(CH₂)ₙ-(4- to 8-membered heterocycle containing from 1 to 3 ring heteroatoms selected from O, S and N) and —(CH₂)ₙ-(4- to 8-membered heterocycle containing from 1 to 3 ring heteroatoms selected from O, S and N) groups being further optionally substituted by from 1 to 4 groups selected from halo, CN, —CH₂-CN, —CH₃, —CH₂F, —CHF₂, CF₃, —O—CH₃, —O—CH₂F, —O—CHF₂, or —O—CF₃;
b) the —(CH₂)ₙ-linking group of the R₂ₐ—(CH₂)ₙ—C₃-C₈ cycloalkyl, —(CH₂)ₙ—C₅-C₈ cycloalkenyl, and —(CH₂)ₙ-(4- to 8-membered heterocycle containing from 1 to 3 ring heteroatoms selected from O, S and N) and —(CH₂)ₙ—O-(4- to 8-membered heterocycle containing from 1 to 3 ring heteroatoms selected from O, S and N) groups being further optionally substituted by from 1 to 4 groups selected from halo, CN —CH₂—CN, —CH₃, —CH₂F, —CHF₂, CF₃, —O—CH₃, —O—CH₂F, —O—CHF₂, or —O—CF₃;

with R₂ₐ also optionally being a phenyl or pyridyl group optionally substituted by from 1 to 3 substituents selected from H, CN, —CH₂—CN, halogen, C₁-C₃ alkyl, —CH₂F, —CHF₂, CF₃, —O—C₁-C₃ alkyl, —O—CH₂F, —O—CHF₂, or —O—CF₃; and

R_{2b} and R_{2c} are independently selected from H, halogen, CN, —CH₂—CN, C₁-C₃ alkyl, C₁-C₃ alkoxy, —CH₂F, —CHF₂, CF₃, —O—CH₂F, —O—CHF₂, or —O—CF₃;

n in each instance is an integer independently selected from 1, 2 or 3;

or a pharmaceutically acceptable salt thereof.

The invention is also comprises pharmaceutical compositions comprising a therapeutically effective amount of a compound herein, or a pharmaceutically acceptable salt thereof, and a pharmaceutically acceptable carrier.

The invention is also directed, in part, to methods of treating FAAH-mediated diseases or conditions including acute pain, chronic pain, neuropathic pain, nociceptive pain, inflammatory pain, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, or cardiovascular disease in a subject by administering to a subject in need thereof a therapeutically effective amount of one or more of the compounds herein, or a pharmaceutically acceptable salt thereof.

### DETAILED DESCRIPTION

This invention also includes compounds of Formula I, above, wherein R_{2c} is hydrogen and X, Y, Z, R₁, R₂ₐ, R_{2b}, and n are as defined above, or a pharmaceutically acceptable salt thereof.

Within each of the groups of compounds described herein are subsets of compounds wherein Ar is selected from the group of isoxazole, 1,2,4-triazine, 1,3,4-oxadiazole, 1,2,4-oxadiazole, pyrazole, 1,2,3-triazole, 1,2,4-triazole, or tetrazole; and X, Y, Z, n, R₁, R₂ₐ, R_{2b}, and R_{2c} are as defined above; or a pharmaceutically acceptable salt thereof.

Other compounds within each of the groups of compounds described herein are those wherein Ar is isoxazole; and X, Y, Z, n, R₁, R₂ₐ, R_{2b}, and R_{2c} are as defined above; or a pharmaceutically acceptable salt thereof.

Within each of the groups of compounds described above are further groups of compounds wherein:

Ar is isoxazole, 1,2,4-triazine, 1,3,4-oxadiazole, 1,2,4-oxadiazole, pyrazole, 1,2,3-triazole, 1,2,4-triazole, or tetrazole;

R₁ is selected from H, C₁-C₆ alkyl, C₁-C₆ alkoxy, halogen, C₁-C₃ haloalkyl, C₁-C₃ haloalkoxy, C₃-C₆ cycloalkyl, or CN;

R₂ₐ is H, C₁-C₆ alkyl, C₁-C₆ alkoxy, C₂-C₆ alkenyl, halogen, C₁-C₃ haloalkyl, C₁-C₃ haloalkoxy, C₃-C₆ cycloalkyl, —(CH₂)ₙ—C₃-C₆ cycloalkyl, C₃-C₆ cycloalkoxy, C₅-C₆ cycloalkenyl, —(CH₂)ₙ—C₅-C₆ cycloalkenyl,C₆-C₆ cycloalkenyloxy, 4- to 6-membered oxygen-containing heterocycle, —(CH₂)ₙ—O-(4- to 6-membered oxygen-containing heterocycle), —(CH₂)ₙ—O-(4- to 6-membered oxygen-containing heterocycle) or CN;

R_{2b} and R_{2c} are independently selected from H, halogen, C₁-C₃ alkyl, C₁-C₃ alkoxy, —CH₂F, —CHF₂, CF₃, —O—CH₂F, —O—CHF₂, or —O—CF₃;

n in each instance is an integer independently selected from 1, 2 or 3;

or a pharmaceutically acceptable salt thereof.

Within each of the groups of compounds described above are also provided further groups of compounds wherein:

Ar is isoxazole;

R₁ is selected from H, C₁-C₆ alkyl, C₁-C₆ alkoxy, halogen, C₁-C₃ haloalkyl, C₁-C₃ haloalkoxy, C₃-C₆ cycloalkyl, or CN; and

R_{2b} and R_{2c} are independently selected from H, halogen, C₁-C₃ alkyl, —CH₂F, —CHF₂, CF₃, C₁-C₃ alkoxy, —O—CH₂F, —O—CHF₂, or —O—CF₃;

n in each instance is an integer independently selected from 1, 2 or 3;

or a pharmaceutically acceptable salt thereof.

Within each of the groups described herein are subgroups of compounds, or the pharmaceutically acceptable salts thereof, wherein the definitions of X, Y and Z are as follows: 1) X is N and Y and Z are N or CH; 2) X is N and Y and Z are CH; 3) X and Y are N and Z is CH; and 4) Z is N and X and Y are CH.

Preferable groups of compounds of formula I are those wherein independently:

R₁ has the value of R₁ of any of the specific compounds mentioned below;

X has the value of X of any of the specific compounds mentioned below;

Y has the value of Y of any of the specific compounds mentioned below;

Z has the value of Z of any of the specific compounds mentioned below;

Ar has the value of Ar of any of the specific compounds mentioned below;

R₂ₐ has the value of R₂ₐ of any of the specific compounds mentioned below;

R_{2b} has the value of R_{2b} of any of the specific compounds mentioned below; and/or

R_{2c} has the value of R_{2c} of any of the specific compounds mentioned below.

The most preferable compounds of formula I are those specificaly mentioned below.

### DEFINITIONS AND ABBREVIATIONS

This disclosure uses the definitions provided below. Some of the chemical formulae may include a dash (“-”) to indicate a bond between atoms or indicate a point of attachment. “Substituted” groups are those in which one or more hydrogen atoms have been replaced with one or more non-hydrogen atoms or groups. “Alkyl” refers to straight chain or branched chain saturated hydrocarbon groups, generally having a specified number of carbon atoms (i.e., C₁-C₆alkyl). “Alkenyl” refers to straight chain or branched chain hydrocarbon groups having one or more unsaturated carbon-carbon double bond, and having a specified number of carbon atoms (i.e., C₂-C₆alkenyl). Examples of alkenyl groups include ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-buten-2-yl, 2-methyl-1-propen-1-yl, 2-methyl-2-propen-1-yl, 1,3-butadien-1-yl, 1,3-butadien-2-yl, and the like. “Alkynyl” refers to straight chain or branched chain hydrocarbon groups having one or more carbon-carbon triple bond, and having a specified number of carbon atoms (i.e., C₂-C₆ alkynyl) . Examples of alkynyl groups include ethynyl, 1-propyn-1-yl, 2-propyn-1-yl, 1-butyn-1-yl, 3-butyn-1-yl, 3-butyn-2-yl, 2-butyn-1-yl, and the like.

“Alkoxy” refers to alkyl-O- groups wherein the alkyl portions, which may be straight chain or branched, have from 1 to 6 carbon atoms. Examples of alkoxy groups include methoxy, ethoxy, n-propoxy, i-propoxy, n-butoxy, s-butoxy, t-butoxy, n-pentoxy, s-pentoxy, and the like.

“Halo,” or “halogen” may be used interchangeably, and are fluoro, chloro, bromo, and iodo. The terms “haloalkyl” or “-O-haloalkyl” refer, respectively, to alkyl or alkoxy groups substituted by one or more halogens. Examples include —CF₃, —CH₂—CF₃, —CF₂—CF₃, —O—CF₃, and —OCH₂—CF₃.

“Cycloalkyl” refers to saturated monocyclic and bicyclic hydrocarbon rings, generally having a specified number of carbon atoms that comprise the ring (i.e. C₃-C₈ cycloalkyl). The cycloalkyl groups may include one or more substituents. Examples of monocyclic cycloalkyl groups include cyclopropyl, cyclobutyl, cyclopentyl, cyclohexyl, and the like. Examples of bicyclic cycloalkyl groups include bicyclo[1.1.0]butyl, bicyclo[1.1.1]pentyl, bicyclo[2.1.0]pentyl, bicyclo[2.1.1]hexyl, bicyclo[3.1.0]hexyl, bicyclo[2.2.1]heptyl, bicyclo[3.2.0]heptyl, bicyclo[3.1.1]heptyl, bicyclo[4.1.0]heptyl, bicyclo[2.2.2]octyl, bicyclo[3.2.1]octyl, bicyclo[4.1.1]octyl, bicyclo[3.3.0]octyl, bicyclo[4.2.0]octyl, and the like.

“Cycloalkenyl” refers monocyclic and bicyclic hydrocarbon rings having one or more carbon-carbon double bonds, generally having a specified number of carbon atoms that comprise the ring (i.e., C₅-C₇ cycloalkenyl), such as cyclopentene, cyclohexene, cycloheptene or cyclooctene groups. Useful substituents include alkyl, alkenyl, alkynyl, haloalkyl, haloalkenyl, haloalkynyl, alkoxy, alkoxycarbonyl, alkanoyl, and halo, as defined above, and hydroxy, mercapto, nitro, and amino and the like.

“Cycloalkoxy” and “cycloalkenyloxy” refer, respectively, to cycloalkyl-O- and cycloalkenyl-O-, wherein cycloalkyl and cycloalkenyl are defined above. References to cycloalkoxy and “cycloalkenyloxy” generally include a specified number of carbon atoms. Examples of cycloalkoxy groups include cyclopropoxy, cyclobutoxy, cyclopentoxy, and cyclohexoxy groups. Examples of cycloalkenyloxy groups include , 1-cyclopentenoxy, 2-cyclopentenoxy, 3-cyclopentenoxy, 1-cyclohexenoxy, 2-cyclohexenoxy, 3-cyclohexenoxy, and the like.

“Heterocycle” refers to 4- to 8-membered monocyclic or bicyclic rings which are fully or partially saturated and contain from 1 to 3 ring heteroatoms selected from O, S or N. Examples of heterocyclic rings include azetidine, oxirane, oxetane, tetrahydrothiophene, furan, tetrahydrofuran, dihydrofuran, 1,3-dioxolane, tetrahydropyran, dioxane, pyrrolidine, isothiazolidine, pyran, dihydropyran, piperidine, morpholine, azepane, and diazepane, The rings may also be bound through a —(CH₂)ₙ- or —(CH₂)ₙ—O- linking group wherein n is an integer selected from 1, 2 or 3. Some compounds herein contain 4- to 6-membered oxygen-containing heterocycle groups , including oxetane, tetrahydrofuran, furan, dihydrofuran, pyran, dihydropyran, tetrahydropyran, and dioxane.

“Aryl” and “arylene” refer to monocyclic or bicyclic monovalent and divalent aromatic carbocyclic groups, such as phenyl, biphenyl or naphthyl groups.

“Heteroaryl” and “heteroarylene” refer to monovalent or divalent aromatic groups, respectively, containing from 1 to 4 ring heteroatoms selected from O, S or N. Examples of monocyclic (and monovalent) heteroaryl groups include pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, and the like. 5-membered heteroaryl moieties include furan, thiophene, pyrrole, imidazole, pyrazole, triazole, tetrazole, thiazole, oxazole, isoxazole, oxadiazole, thiadiazole, and isothiazole groups, including the various isomers thereof.

Heteroaryl and heteroarylene groups also include bicyclic groups, tricyclic groups, including fused ring systems wherein at least one ring is aromatic. Examples of multicyclic (and monovalent) aryl groups include pyrenyl, carbazolyl, benzofuranyl, benzothiophenyl, indolyl, benzoxazolyl, benzodioxazolyl, benzimidazolyl, indazolyl, benzotriazolyl, benzothiofuranyl, benzothiazolyl, benzotriazolyl, benzotetrazolyl benzoisoxazolyl, benzoisothiazolyl, benzoimidazolinyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, indazolyl, purinyl, indolizinyl, imidazo[1,2-a]pyridinyl, imidazo[1,5-a]pyridinyl, pyrazolo[1,5-a]pyridinyl, pyrrolo[1,2-b]pyridinyl, and imidazo[1,2-c]pyridinyl. Other examples include quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, pyrimido[4,5-d]pyrimidinyl, phenanthridinyl, phenanthrolinyl, phenazinyl, phenothiazinyl, phenoxathiinyl, phenoxazinyl, acridinyl, azocinyl, 4aH-carbazolyl, chromanyl, chromenyl, indolenyl, indolinyl, 3H-indolyl, isobenzofuranyl, isochromanyl, isoindazolyl, isoindolinyl, pyrimidinyl, pteridinyl, phthalazinyl, purinyl, pyridazinyl, pyrazinyl, pyridooxazole, pyridoimidazole, pyridothiazole, pyridyl, pyridopyrimidinyl, quinoxalinyl, quinazolinyl, thianthrenyl, xanthenyl, and the like.

“Subject” refers to a mammal, including humans. “Treating” refers to reversing, alleviating, inhibiting the progress of, or preventing a disorder or condition to which such term applies, or to reversing, alleviating, inhibiting the progress of, or preventing one or more symptoms of such disorder or condition. “Therapeutically effective amount” refers to the quantity of a compound that may be used for treating a subject, which amount may depend on the weight and age of the subject and the route of administration, among other things. “Excipient” or “adjuvant” refers to any substance in a pharmaceutical formulation that is not an active pharmaceutical ingredient (API). “Pharmaceutical composition” refers to the combination of one or more drug substances and one or more excipients. “Drug product,” “pharmaceutical dosage form,” “dosage form,” “final dosage form” and the like, refer to a pharmaceutical composition that is administered to a subject in need of treatment and generally may be in the form of tablets, capsules, liquid solutions or suspensions, patches, films, and the like.

The present invention relates to compounds of Formula I, and compounds specifically named below, and their pharmaceutically acceptable salts, which are effective for inhibiting the activity of FAAH. The invention also concerns materials and methods for preparing the compounds, pharmaceutically acceptable salts, pharmaceutical compositions containing them and one or more pharmaceutically acceptable carriers and/or excipients, and their use for treating a variety of disorders such as pain, depression, or anxiety.

The compounds herein and the pharmaceutically acceptable salts thereof, which includes those of Formula I, may be used to treat pain (including neuropathic pain, nociceptive pain, and inflammatory pain); urinary incontinence; overactive bladder; emesis; movement disorders; glaucoma; psoriasis; multiple sclerosis; cerebrovascular disorders; brain injury; gastrointestinal disorders; hypertension; cardiovascular disease; and central nervous system disorders including anxiety, depression, sleeping disorders, and eating disorders.

Physiological pain is an important protective mechanism designed to warn of danger from potentially injurious stimuli from the external environment. The system operates through a specific set of primary sensory neurons and is activated by noxious stimuli via peripheral transducing mechanisms (see Millan, 1999, Prog. Neurobiol., 57, 1-164 for a review). These sensory fibers are known as nociceptors and are characteristically small diameter axons with slow conduction velocities. Nociceptors encode the intensity, duration and quality of noxious stimulus and by virtue of their topographically organized projection to the spinal cord, the location of the stimulus. The nociceptors are found on nociceptive nerve fibers of which there are two main types, A-delta fibers (myelinated) and C fibers (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a heightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibers associated with maladaptation and aberrant activity (Woolf & Salter, 2000, Science, 288, 1765-1768).

Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994, Textbook of Pain, 13-44). The activation of nociceptors activates two types of afferent nerve fibers. Myelinated A-delta fibers transmit rapidly and are responsible for sharp and stabbing pain sensations, while unmyelinated C fibers transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumor related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain is currently defined as pain initiated or caused by a primary lesion or dysfunction in the nervous system. Nerve damage can be caused by trauma and disease and thus the term ‘neuropathic pain’ encompasses many disorders with diverse etiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact etiology of rheumatoid arthritis is unknown, but current hypotheses suggest that both genetic and microbiological factors may be important (Grennan & Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, 2002, Ann Pharmacother., 36, 679-686; McCarthy et al., 1994, Textbook of Pain, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhea, cystitis and pancreatitis and pelvic pain.

It should be noted that some types of pain have multiple etiologies and thus can be classified in more than one area, e.g. back pain and cancer pain have both nociceptive and neuropathic components. Other types of pain include pain resulting from musculo-skeletal disorders, including myalgia, fibromyalgia spondylitis, sero-negative (non-rheumatoid) arthropathies, non-articular rheumatism, dystrophinopathy glycogenolysis, polymyositis and pyomyositis; heart and vascular pain, including pain caused by angina myocardical infarction, mitral stenosis, pericarditis, Raynaud's phenomenon, scleredoma and skeleta muscle ischemia; head pain, such as migraine (including migraine with aura and migraine without aura) cluster headache, tension-type headache mixed headache and headache associated with vascular disorders; and orofacial pain, including dental pain, otic pain, burning mouth syndrome and temporomandibular myofascial pain.

As described above, the compounds herein, and the pharmaceutically acceptable salts thereof, may be used to treat CNS disorders, including schizophrenia and other psychotic disorders, mood disorders, anxiety disorders, sleep disorders, and cognitive disorders, such as delirium, dementia, and amnestic disorders. The standards for diagnosis of these disorders may be found in the American Psychiatric Association's Diagnostic and Statistical Manual of Mental Disorders (4th ed., 2000), which is commonly referred to as the DSM Manual.

For the purposes of this disclosure, schizophrenia and other psychotic disorders include schizophreniform disorder, schizoaffective disorder, delusional disorder, brief psychotic disorder, shared psychotic disorder, psychotic disorder due to general medical condition, and substance-induced psychotic disorder, as well as medication-induced movement disorders, such as neuroleptic-induced Parkinsonism, neuroleptic malignant syndrome, neuroleptic-induced acute dystonia, neuroleptic-induced acute akathisia, neuroleptic-induced tardive dyskinesia, and medication-induced postural tremor.

Mood disorders include depressive disorders, such as major depressive disorder, dysthymic disorder, premenstrual dysphoric disorder, minor depressive disorder, recurrent brief depressive disorder, postpsychotic depressive disorder of schizophrenia, and major depressive episode with schizophrenia;

bipolar disorders, such as bipolar I disorder, bipolar II disorder, cyclothymia, and bipolar disorder with schizophrenia; mood disorders due to general medical condition; and substance-induced mood disorders.

Anxiety disorders include panic attack, agoraphobia, panic disorder without agoraphobia, agoraphobia without history of panic disorder, specific phobia, social phobia (social anxiety disorder), obsessive-compulsive disorder, posttraumatic stress disorder, acute stress disorder, generalized anxiety disorder, anxiety disorder due to general medical condition, substance-induced anxiety disorder, and mixed anxiety-depressive disorder.

Sleep disorders include primary sleep disorders, such as dyssomnias (primary insomnia, primary hypersomnia, narcolepsy, breathing-related sleep disorder, circadian rhythm sleep disorder, sleep deprivation, restless legs syndrome, and periodic limb movements) and parasomnias (nightmare disorder, sleep terror disorder, sleepwalking disorder, rapid eye movement sleep behavior disorder, and sleep paralysis); sleep disorders related to another mental disorder, including insomnia related to schizophrenia, depressive disorders, or anxiety disorders, or hypersomnia associated with bipolar disorders; sleep disorders due to a general medical condition; and substance-induced sleep disorders,

Delirium, dementia, and amnestic and other cognitive disorders, includes delirium due to a general medical condition, substance-induced delirium, and delirium due to multiple etiologies; dementia of the Alzheimer's type, vascular dementia, dementia due to general medical conditions, dementia due to human immunodeficiency virus disease, dementia due to head trauma, dementia due to Parkinson's disease, dementia due to Huntington's disease, dementia due to Pick's disease, dementia due to Creutzfeldt-Jakot disease, dementia due to other general medical conditions, substance-induced persisting dementia, dementia due to multiple etiologies; amnestic disorders due to a general medical condition, and substance-induced persisting amnestic disorder.

Substance-induced disorders refer to those resulting from the using, abusing, dependence on, or withdrawal from, one or more drugs or toxins, including alcohol, amphetamines or similarly acting sympathomimetics, caffeine, cannabis, cocaine, hallucinogens, inhalants, nicotine, opioids, phencyclidine or similarly acting arylcyclohexylamines, and sedatives, hypnotics, or anxiolytics, among others.

Urinary incontinence includes the involuntary or accidental loss of urine due to the inability to restrain or control urinary voiding. Urinary incontinence includes mixed urinary incontinence, nocturnal enuresis, overflow incontinence, stress incontinence, transient urinary incontinence, and urge incontinence.

The compounds described and specifically named herein may form pharmaceutically acceptable complexes, salts, solvates and hydrates. The salts include acid addition salts (including di-acids) and base salts.

Pharmaceutically acceptable acid addition salts include salts derived from inorganic acids such as hydrochloric acid, nitric acid, phosphoric acid, sulfuric acid, hydrobromic acid, hydroiodic acid, hydrofluoric acid, and phosphorous acids, as well salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include acetate, adipate, aspartate, benzoate besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride, chloride, hydrobromide, bromide, hydroiodide, iodide, isethionate, lactate, malate, maleate malonate, mesylate, methylsulfate, naphthylate, 2-napsylate, nicotinate, nitrate, orotate, oxalate, almitate, pamoate, phosphate, hydrogen phosphate, dihydrogen phosphate, pyroglutamate, saccharate, stearate, succinate, tannate, tartrate, tosylate, trifluoroacetate and xinofoate salts.

Pharmaceutically acceptable base salts include salts derived from bases, including metal cations, such as an alkali or alkaline earth metal cation, as well as amines. Examples of suitable metal cations include sodium (Na⁺), potassium (K⁺), magnesium (Mg²⁺), calcium (Ca²⁺), zinc (Zn²⁺), and aluminum (Al³⁺). Examples of suitable amines include arginine, N,N′-dibenzylethylenediamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine. For a discussion of useful acid addition and base salts, see S. M. Berge et al., “Pharmaceutical Salts,” 66 J. Pharm. Sci., 1-19 (1977); see also Stahl and Wermuth, Handbook of Pharmaceutical Salts: Properties, Selection, and Use (2002).

Pharmaceutically acceptable salts may be prepared using various methods. For example, one may react a compound with an appropriate acid or base to give the desired salt. One may also react a precursor of the compound with an acid or base to remove an acid- or base-labile protecting group or to open a lactone or lactam group of the precursor. Additionally, one may convert a salt of the compound to another salt through treatment with an appropriate acid or base or through contact with an ion exchange resin. Following reaction, one may then isolate the salt by filtration if it precipitates from solution, or by evaporation to recover the salt. The degree of ionization of the salt may vary from completely ionized to almost non-ionized.

The compounds herein, and the pharmaceutically acceptable salts thereof, may exist in a continuum of solid states ranging from fully amorphous to fully crystalline. They may also exist in unsolvated and solvated forms. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent may be isotopically substituted (e.g., D₂O, d₆-acetone, d₆-DMSO).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will be the norm.

The compounds herein, and the pharmaceutically acceptable salts thereof, may also exist as multi-component complexes (other than salts and solvates) in which the compound and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together. See, e.g., O. Almarsson and M. J. Zaworotko, Chem. Commun., 17:1889-1896 (2004). For a general review of multi-component complexes, see J. K. Haleblian, J. Pharm. Sci. 64(8):1269-88 (1975).

“Prodrugs” refer to compounds that when metabolized in vivo, undergo conversion to compounds having the desired pharmacological activity. Prodrugs may be prepared by replacing appropriate functionalities present in pharmacologically active compounds with “pro-moieties” as described, for example, in H. Bundgaar, Design of Prodrugs (1985). Examples of prodrugs include ester, ether or amide derivatives of the compounds herein, and their pharmaceutically acceptable salts. For further discussions of prodrugs, see e.g., T. Higuchi and V. Stella “Pro-drugs as Novel Delivery Systems,” ACS Symposium Series 14 (1975) and E. B. Roche ed., Bioreversible Carriers in Drug Design (1987).

“Metabolites” refer to compounds formed in vivo upon administration of pharmacologically active compounds. Examples include hydroxymethyl, hydroxy, secondary amino, primary amino, phenol, and carboxylic acid derivatives of compounds herein, and the pharmaceutically acceptable salts thereof having methyl, alkoxy, tertiary amino, secondary amino, phenyl, and amide groups, respectively.

Geometrical (cis/trans) isomers may be separated by conventional techniques such as chromatography and fractional crystallization.

“Tautomers” refer to structural isomers that are interconvertible via a low energy barrier. Tautomeric isomerism (tautomerism) may take the form of proton tautomerism in which the compound contains, for example, an imino, keto, or oxime group, or valence tautomerism in which the compound contains an aromatic moiety.

Compounds described herein also include all pharmaceutically acceptable isotopic variations, in which at least one atom is replaced by an atom having the same atomic number, but an atomic mass different from the atomic mass usually found in nature. Isotopes suitable for inclusion in the compounds herein, and the pharmaceutically acceptable salts thereof include, for example, isotopes of hydrogen, such as ²H and ³H; isotopes of carbon, such as ¹¹C, ¹³C and ¹⁴C; isotopes of nitrogen, such as ¹³N and ¹⁵N; isotopes of oxygen such as ¹⁵O, ¹⁷O and ¹⁸O; isotopes of sulfur, such as ³⁵S; isotopes of fluorine, such as ¹⁸F; isotopes of chlorine, such as ³⁶Cl, and isotopes of iodine, such as ¹²³I and ¹²⁵I. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies. Substitution with positron emitting isotopes, such as ¹¹C, ¹⁸F, ¹⁵O and ¹³N, may be useful in Positron Emission Topography (PET) studies for examining substrate receptor occupancy. Isotopically-labelled compounds may be prepared by processes analogous to those described elsewhere in the disclosure using an appropriate isotopically-labelled reagent in place of a non-labelled reagent.

The compounds herein, and the pharmaceutically acceptable salts thereof, can be administered as crystalline or amorphous forms, prodrugs, metabolites, hydrates, solvates, complexes, and tautomers thereof, as well as all isotopically-labelled compounds thereof. They may be administered alone or in combination with one another or with one or more pharmacologically active compounds which are different than the compounds described or specifically named herein, and the pharmaceutically acceptable salts thereof. Generally, one or more these compounds are administered as a pharmaceutica composition (a formulation) in association with one or more pharmaceutically acceptable excipients. The choice of excipients depends on the particular mode of administration, the effect of the excipient on solubility and stability, and the nature of the dosage form, among other things. Useful pharmaceutical compositions and methods for their preparation may be found, for example, in A. R. Gennaro (ed.), Remington: The Science and Practice of Pharmacy (20th ed., 2000).

Also provided herein are pharmaceutical compositions comprising a therapeutically effective amount of a compound described herein, or a pharmaceutically acceptable salt thereof, and on or more pharmaceutically acceptable carriers and/or excipients. The compounds herein, and the pharmaceutically acceptable salts thereof, may be administered orally. Oral administration may involve swallowing in which case the compound enters the bloodstream via the gastrointestinal tract. Alternatively or additionally, oral administration may involve mucosal administration (e.g., buccal, sublingual, supralingual administration) such that the compound enters the bloodstream through the oral mucosa. Formulations suitable for oral administration include solid, semi-solid and liquid systems such as tablets; soft or hard capsules containing multi- or nano-particulates, liquids, or powders; lozenges which may be liquid-filled; chews; gels; fast dispersing dosage forms; films; ovules; sprays; and buccal or mucoadhesive patches.

Liquid formulations include suspensions, solutions, syrups and elixirs. Such formulations may be employed as fillers in soft or hard capsules (made, for example, from gelatin or hydroxypropyl methylcellulose) and typically comprise a carrier (e.g., water, ethanol, polyethylene glycol, propylene glycol, methylcellulose, or a suitable oil) and one or more emulsifying agents, suspending agents or both. Liquid formulations may also be prepared by the reconstitution of a solid (e.g., from a sachet).

The compounds herein, and the pharmaceutically acceptable salts thereof, may also be used in fast-dissolving, fast-disintegrating dosage forms such as those described in Liang and Chen, Expert Opinion in Therapeutic Patents, 11(6):981-986 (2001).

For tablet dosage forms, depending on dose, the active pharmaceutical ingredient (API) may comprise from about 1 wt % to about 80 wt % of the dosage form or more typically from about 5 wt % to about 60 wt % of the dosage form. In addition to the API, tablets may include one or more disintegrants, binders, diluents, surfactants, glidants, lubricants, anti-oxidants, colorants, flavoring agents, preservatives, and taste-masking agents. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, C₁-C₆ alkyl-substituted hydroxypropylcellulose, starch, pregelatinized starch, and sodium alginate. Generally, the disintegrant will comprise from about 1 wt % to about 25 wt % or from about 5 wt % to about 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropylcellulose and hydroxypropylmethylcellulose. Tablets may also contain diluents, such as lactose (monohydrate, spray-dried monohydrate, anhydrous), mannitol, xylitol, dextrose, sucrose, sorbitol, microcrystalline cellulose, starch and dibasic calcium phosphate dihydrate. Tablets may also include surface active agents, such as sodium lauryl sulfate and polysorbate 80, and glidants such as silicon dioxide and talc. When present, surface active agents may comprise from about 0.2 wt % to about 5 wt % of the tablet, and glidants may comprise from about 0.2 wt % to about 1 wt % of the tablet. Tablets may also contain lubricants such as magnesium stearate, calcium stearate, zinc stearate, sodium stearyl fumarate, and mixtures of magnesium stearate with sodium lauryl sulfate. Lubricants may comprise from about 0.25 wt % to about 10 wt % or from about 0.5 wt % to about 3 wt % of the tablet. Tablet blends may be compressed directly or by roller compaction to form tablets. Tablet blends or portions of blends may alternatively be wet-, dry-, or melt-granulated, melt congealed, or extruded before tableting. If desired, prior to blending one or more of the components may be sized by screening or milling or both. The final dosage form may comprise one or more layers and may be coated, uncoated, or encapsulated. Exemplary tablets may contain up to about 80 wt % of API, from about 10 wt % to about 90 wt % of binder, from about 0 wt % to about 85 wt % of diluent, from about 2 wt % to about 10 wt % of disintegrant, and from about 0.25 wt % to about 10 wt % of lubricant. For a discussion of blending, granulation, milling, screening, tableting, coating, as well as a description of alternative techniques for preparing drug products, see A. R. Gennaro (ed.), Remington: The Science and Practice of Pharmacy (20th ed., 2000); H. A. Lieberman et al. (ed.), Pharmaceutical Dosage Forms: Tablets, Vol. 1-3 (2d ed., 1990); and D. K. Parikh & C. K. Parikh, Handbook of Pharmaceutical Granulation Technology, Vol. 81 (1997).

Consumable oral films for human or veterinary use are pliable water-soluble or water-swellable thin film dosage forms which may be rapidly dissolving or mucoadhesive. In addition to the API, a typical film includes one or more film-forming polymers, binders, solvents, humectants, plasticizers, stabilizers or emulsifiers, viscosity-modifying agents, and solvents. Other film ingredients may include anti-oxidants, colorants, flavorants and flavor enhancers, preservatives, salivary stimulating agents, cooling agents, co-solvents (including oils), emollients, bulking agents, anti-foaming agents, surfactants, and taste-masking agents. Some components of the formulation may perform more than one function. In addition to dosing requirements, the amount of API in the film may depend on its solubility. If water soluble, the API would typically comprise from about 1 wt % to about 80 wt % of the non-solvent components (solutes) in the film or from about 20 wt % to about 50 wt % of the solutes in the film. A less soluble API may comprise a greater proportion of the composition, typically up to about 88 wt % of the non-solvent components in the film.

The film-forming polymer may be selected from natural polysaccharides, proteins, or synthetic hydrocolloids and typically comprises from about 0.01 wt % to about 99 wt % or from about 30 wt % to about 80wt % of the film. Film dosage forms are typically prepared by evaporative drying of thin aqueous films coated onto a peelable backing support or paper, which may carried out in a drying oven or tunnel (e.g., in a combined coating-drying apparatus), in lyophilization equipment, or in a vacuum oven.

Useful solid formulations for oral administration may include immediate release formulations and modified release formulations. Modified release formulations include delayed-, sustained-, pulsed-, controlled-, targeted-, and programmed-release. For a general description of suitable modified release formulations, see U.S. Pat. No. 6,106,864. For details of other useful release technologies, such as high energy dispersions and osmotic and coated particles, see Verma et al, Pharmaceutical Technology On-line (2001) 25(2):1-14. Compounds herein, and the pharmaceutically acceptable salts thereof, may also be administered directly into the blood stream, muscle, or an internal organ of the subject. Suitable techniques for parenteral administration include intravenous, intraarterial, intraperitoneal, intrathecal, intraventricular, intraurethral, intrasternal, intracranial, intramuscular, intrasynovial, and subcutaneous administration. Suitable devices for parenteral administration include needle injectors, including microneedle injectors, needle-free injectors, and infusion devices.

Parenteral formulations are typically aqueous solutions which may contain excipients such as salts, carbohydrates and buffering agents (e.g., pH of from about 3 to about 9). For some applications, however, the compounds herein, and the pharmaceutically acceptable salts thereof, may be more suitably formulated as a sterile non-aqueous solution or as a dried form to be used in conjunction with a suitable vehicle such as sterile, pyrogen-free water. The preparation of parenteral formulations under sterile conditions (e.g., by lyophilization) may be readily accomplished using standard pharmaceutical techniques.

The solubility of compounds which are used in the preparation of parenteral solutions may be increased through appropriate formulation techniques, such as the incorporation of solubility-enhancing agents. Formulations for parenteral administration may be formulated to be immediate or modified release. Modified release formulations include delayed, sustained, pulsed, controlled, targeted, and programmed release. Thus, compounds herein, and the pharmaceutically acceptable salts thereof, may be formulated as a suspension, a solid, a semi-solid, or a thixotropic liquid for administration as an implanted depot providing modified release of the active compound. Examples of such formulations include drug-coated stents and semi-solids and suspensions comprising drug-loaded poly(DL-lactic-coglycolic)acid (PGLA) microspheres.

The compounds herein, and the pharmaceutically acceptable salts thereof, may also be administered topically, intradermally, or transdermally to the skin or mucosa. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers may include alcohol, water, mineral oil, liquid petrolatum, white petrolatum, glycerin, polyethylene glycol and propylene glycol. Topical formulations may also include penetration enhancers. See, e.g., Finnin and Morgan, J. Pharm. Sci. 88(10):955-958 (1999). Other means of topical administration include delivery by electroporation, iontophoresis, phonophoresis, sonophoresis and microneedle or needle-free injection. Formulations for topical administration may be formulated to be immediate or modified release as described above.

The compounds herein, and the pharmaceutically acceptable salts thereof, may also be administered intranasally or by inhalation, typically in the form of a dry powder, an aerosol spray, or nasal drops. An inhaler may be used to administer the dry powder, which comprises the API alone, a powder blend of the API and a diluent, such as lactose, or a mixed component particle that includes the API and a phospholipid, such as phosphatidylcholine. For intranasal use, the powder may include a bioadhesive agent, e.g., chitosan or cyclodextrin. A pressurized container, pump, sprayer, atomizer, or nebulizer, may be used to generate the aerosol spray from a solution or suspension comprising the API, one or more agents for dispersing, solubilizing, or extending the release of the API (e.g., EtOH with or without water), one or more solvents (e.g., 1,1,1,2-tetrafluoroethane or 1,1,1,2,3,3,3-heptafluoropropane) which serve as a propellant, and an optional surfactant, such as sorbitan trioleate, oleic acid, or an oligolactic acid. An atomizer using electrohydrodynamics may be used to produce a fine mist.

Prior to use in a dry powder or suspension formulation, the drug product is usually comminuted to a particle size suitable for delivery by inhalation (typically 90% of the particles, based on volume, having a largest dimension less than 5 microns). This may be achieved by any appropriate size reduction method such as spiral jet milling, fluid bed jet milling, supercritical fluid processing, high pressure homogenization, or spray drying.

Capsules, blisters and cartridges (made, for example, from gelatin or hydroxypropylmethyl cellulose) for use in an inhaler or insufflator may be formulated to contain a powder mixture of the active compound, a suitable powder base such as lactose or starch, and a performance modifier such as L-leucine, mannitol, or magnesium stearate. The lactose may be anhydrous or monohydrated. Other suitable excipients include dextran, glucose, maltose, sorbitol, xylitol, fructose, sucrose, and trehalose.

A suitable solution formulation for use in an atomizer using electrohydrodynamics to produce a fine mist may contain from about 1 μg to about 20 mg of the API per actuation and the actuation volume may vary from about 1 μL to about 100 μL. A typical formulation may comprise one or more of the compounds herein, or a pharmaceutically acceptable salt thereof, propylene glycol, sterile water, EtOH, and NaCl. Alternative solvents, which may be used instead of propylene glycol, include glycerol and polyethylene glycol.

Formulations for inhaled administration, intranasal administration, or both, may be formulated to be immediate or modified release using, for example, PGLA. Suitable flavors, such as menthol and levomenthol, or sweeteners, such as saccharin or sodium saccharin, may be added to formulations intended for inhaled/intranasal administration.

In the case of dry powder inhalers and aerosols, the dosage unit is determined by means of a valve that delivers a metered amount. Units are typically arranged to administer a metered dose or “puff” containing from about 10 μg to about 1000 μg of the API. The overall daily dose will typically range from about 100 μg to about 10 mg which may be administered in a single dose or, more usually, as divided doses throughout the day.

The active compounds may be administered rectally or vaginally, e.g., in the form of a suppository, pessary, or enema. Cocoa butter is a traditional suppository base, but various alternatives may be used as appropriate. Formulations for rectal or vaginal administration may be formulated to be immediate or modified release as described above.

The compounds herein, and the pharmaceutically acceptable salts thereof, and the pharmaceutically acceptable salts thereof may also be administered directly to the eye or ear, typically in the form of drops of a micronized suspension or solution in isotonic, pH-adjusted, sterile saline. Other formulations suitable for ocular and aural administration include ointments, gels, biodegradable implants (e.g. absorbable gel sponges, collagen), non-biodegradable implants (e.g. silicone), wafers, lenses, and particulate or vesicular systems, such as niosomes or liposomes. The formulation may include one or more polymers and a preservative, such as benzalkonium chloride. Typical polymers include crossed-linked polyacrylic acid, polyvinylalcohol, hyaluronic acid, cellulosic polymers (e.g., hydroxypropylmethylcellulose, hydroxyethylcellulose, methyl cellulose), and heteropolysaccharide polymers (e.g., gelan gum). Such formulations may also be delivered by iontophoresis. Formulations for ocular or aural administration may be formulated to be immediate or modified release as described above.

As noted above, the compounds herein, and the pharmaceutically acceptable salts thereof, and their pharmaceutically active complexes, solvates and hydrates, may be combined with one another or with one or more other active pharmaceutically active compounds to treat various diseases, conditions and disorders. In such cases, the active compounds may be combined in a single dosage form as described above or may be provided in the form of a kit which is suitable for coadministration of the compositions. The kit comprises (1) two or more different pharmaceutical compositions, at least one of which contains a compound of Formula I; and (2) a device for separately retaining the two pharmaceutical compositions, such as a divided bottle or a divided foil packet. An example of such a kit is the familiar blister pack used for the packaging of tablets or capsules. The kit is suitable for administering different types of dosage forms (e.g., oral and parenteral) or for administering different pharmaceutical compositions at separate dosing intervals, or for titrating the different pharmaceutical compositions against one another. To assist with patient compliance, the kit typically comprises directions for administration and may be provided with a memory aid.

For administration to human patients, the total daily dose of the claimed and disclosed compounds is typically in the range of about 0.1 mg to about 3000 mg depending on the route of administration. For example, oral administration may require a total daily dose of from about 1 mg to about 3000 mg, while an intravenous dose may only require a total daily dose of from about 0.1 mg to about 300 mg. The total daily dose may be administered in single or divided doses and, at the physician's discretion, may fall outside of the typical ranges given above. Although these dosages are based on an average human subject having a mass of about 60 kg to about 70 kg, the physician will be able to determine the appropriate dose for a patient (e.g., an infant) whose mass falls outside of this weight range.

The claimed and disclosed compounds may be combined with one or more other pharmacologically active compounds for the treatment of one or more related disorders, the pharmacologically active compounds can be selected from: 1) an opioid analgesic, e.g. morphine, fentanyl, codeine, etc.; 2) a nonsteroidal antiinflammatory drug (NSAID), e.g. acetaminophen, aspirin, diclofenac, etodolac, ibuprofen, naproxen, etc.; 3) a barbiturate sedative, e.g. pentobarbital; 4) a benzodiazepine having a sedative action, e.g. diazepam, lorazepam, etc.; 5) an H₁ antagonist having a sedative action, e.g. diphenhydramine; 6) a sedative such as glutethimide, meprobamate, methaqualone or dichloralphenazone; 7) a skeletal muscle relaxant, e.g. baclofen, carisoprodol, chlorzoxazone, cyclobenzaprine, methocarbamol or orphrenadine; 8 an NMDA receptor antagonist; 9) an alpha-adrenergic; 10) a tricyclic antidepressant, e.g. desipramine, imipramine, amitriptyline or nortriptyline; 11) an anticonvulsant, e.g. carbamazepine, lamotrigine, topiratmate or valproate; 12) a tachykinin (NK) antagonist, particularly an NK-3, NK-2 or NK-1 antagonist; 13) a muscarinic antagonist, e.g oxybutynin, tolterodine, etc.; 14) a COX-2 selective inhibitor, e.g. celecoxib, valdecoxib, etc.; 15) a coal-tar analgesic, in particular paracetamol; 16) a neuroleptic such as haloperidol, clozapine, olanzapine, risperidone, ziprasidone, or Miraxion®; 17) a vanilloid receptor (VR1; also known as transient receptor potential channel, TRPV1) agonist (e.g. resinferatoxin) or antagonist (e.g. capsazepine); 18) a beta-adrenergic such as propranolol; 19) a local anaesthetic such as mexiletine; 20) a corticosteroid such as dexamethasone; 21) a 5-HT receptor agonist or antagonist, particularly a 5-HT_{1B/1D} agonist such as eletriptan, sumatriptan, naratriptan, zolmitriptan or rizatriptan; 22) a 5-HT_{2A} receptor antagonist such as R(+)-alpha-(2,3-dimethoxy-phenyl)-1-[2-(4-fluorophenylethyl)]-4-piperidinemethanol (MDL-100907); 23) a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine, or a nicotine partial agonist such as varenicline; 24) Tramadol®; 25) a PDEV inhibitor; 26) an alpha-2-delta ligand such as gabapentin, pregabalin, 3-methylgabapentin, etc.; 27) a cannabinoid receptor (CB1, CB2) ligand, either agonist or antagonist such as rimonabant; 28) metabotropic glutamate subtype 1 receptor (mGluR1) antagonist; 29) a serotonin reuptake inhibitor such as sertraline, sertraline metabolite demethylsertraline, fluoxetine, etc.; 30) a noradrenaline (norepinephrine) reuptake inhibitor, such as buproprion, buproprion metabolite hydroxybuproprion, especially a selective noradrenaline reuptake inhibitor such as reboxetine, in particular (S,S)-reboxetine; 31) a dual serotonin-noradrenaline reuptake inhibitor, such as venlafaxine, O-desmethylvenlafaxine, clomipramine, desmethylclomipramine, duloxetine, milnacipran and imipramine; 32) an inducible nitric oxide synthase (iNOS) inhibitor; 33) an acetylcholinesterase inhibitor such as donepezil; 34) a prostaglandin E₂ subtype 4 (EP4) antagonist; 35) a leukotriene B4 antagonist; 36) a 5-lipoxygenase inhibitor, such as zileuton; 37) a sodium channel blocker, such as lidocaine; 38) a 5-HT3 antagonist, such as ondansetron; or 39) anti-nerve growth factor (NGF) antibodies. It is understood that the pharmaceutical agents just mentioned may be administered in the manner and at the dosages known in the art.

The compounds herein, and the pharmaceutically acceptable salts thereof, may be generally prepared using the techniques described below. Starting materials and reagents may be obtained from commercial sources or may be prepared using literature methods unless otherwise specified.

In some of the reaction schemes and examples below, certain compounds can be prepared using protecting groups, which prevent undesirable chemical reaction at otherwise reactive sites. Protecting groups may also be used to enhance solubility or otherwise modify physical properties of a compound. For a discussion of protecting group strategies, a description of materials and methods for installing and removing protecting groups, and a compilation of useful protecting groups for common functional groups, including amines, carboxylic acids, alcohols, ketones, aldehydes, and the like, see T. W. Greene and P. G. Wuts, Greene's Protective Groups in Organic Chemistry (4^{th} Ed., 2007) and P. Kocienski, Protective Groups (2000).

Generally, the chemical reactions described throughout the specification may be carried out using substantially stoichiometric amounts of reactants, though certain reactions may benefit from using an excess of one or more of the reactants. Additionally, many of the reactions disclosed throughout the specification may be carried out at about room temperature and ambient pressure, but depending on reaction kinetics, yields, and the like, some reactions may be run at elevated pressures or employ higher (e.g., reflux conditions) or lower (e.g., −70° C. to 0° C.) temperatures. Any reference in the disclosure to a stoichiometric range, a temperature range, a pH range, etc., whether or not expressly using the word “range,” also includes the indicated endpoints.

Many of the chemical reactions may also employ one or more compatible solvents, which may influence the reaction rate and yield. Depending on the nature of the reactants, the one or more solvents may be polar protic solvents (including water), polar aprotic solvents, non-polar solvents, or some combination. Representative solvents include saturated aliphatic hydrocarbons (e.g., n-pentane, n-hexane, n-heptane, n-octane); aromatic hydrocarbons (e.g., benzene, toluene, xylenes); halogenated hydrocarbons (e.g., methylene chloride (DCM), chloroform, carbon tetrachloride); aliphatic alcohols (e.g., methanol (MeOH), ethanol (EtOH), propan-1-ol, propan-2-ol (IPA), butan-1-ol, 2-methyl-propan-1-ol, butan-2-ol, 2-methyl-propan-2-ol, pentan-1-ol, 3-methyl-butan-1-ol, hexan-1-ol, 2-methoxy-ethanol, 2-ethoxy-ethanol, 2-butoxy-ethanol, 2-(2-methoxy-ethoxy)-ethanol, 2-(2-ethoxy-ethoxy)-ethanol, 2-(2-butoxy-ethoxy)-ethanol); ethers (e.g., diethyl ether, di-isopropyl ether, dibutyl ether, 1,2-dimethoxy-ethane (DME), 1,2-diethoxy-ethane, 1-methoxy-2-(2-methoxy-ethoxy)-ethane, 1-ethoxy-2-(2-ethoxy-ethoxy)-ethane, tetrahydrofuran (THF), 1,4-dioxane); ketones (e.g., acetone, methyl ethyl ketone (MEK)); esters (methyl acetate, ethyl acetate (EA or EtOAc)); nitrogen-containing solvents (e.g., formamide, N,N-dimethylformamide (DMF), acetonitrile, N-methyl-pyrrolidone (NMP), pyridine, quinoline, nitrobenzene); sulfur-containing solvents (e.g., carbon disulfide, dimethyl sulfoxide (DMSO), tetrahydro-thiophene-1,1,-dioxide); and phosphorus-containing solvents (e.g., hexamethylphosphoric triamide).

The compounds described herein may be present as stereoisomers, such as enantiomers, diastereomers, and geometric isomers (cis/trans olefins). For example, the compounds described herein generally comprise one or more asymmetric carbon atoms and can be present in the form of one or more stereoisomers (e.g., individual enantiomers and mixtures thereof). Additionally, the compounds described herein generally comprise one or more alkenyl moieties and can be present in the form of one or more geometric isomers (e.g., cis/trans or E/Z isomers and mixtures thereof). More specifically, the compounds of the present invention can be present as the 3R,4E isomer, the 3S,4E isomer, the 3R,4Z isomer, the 3S,4Z isomer, or a mixture of two or more of these stereoisomers.

In one embodiment, the compound of Formula I, has the 3R,4E configuration. In another embodiment, the compound of Formula I, has the 3S,4E configuration. In another embodiment, the compound of Formula I, has the 3R,4Z configuration. In another embodiment, the compound of Formula I, has the 3S,4Z configuration.

In another embodiment, the compounds described herein are present as a mixture of two or more stereoisomers selected from the group consisting of the 3R,4E isomer, the 3S,4E isomer, the 3R,4Z isomer, and the 3S,4Z isomer.

The compounds of the present invention (including the precursor intermediates) can have one or more chiral centers and one or more alkenyl moieties. Where the synthesis yields a compound as a mixture of isomers (e.g., enantiomers and/or geometric isomers), the desired isomer (or the desired enantiomerically-enriched mixture or geometrically enriched mixture) can be obtained using conventional chiral resolution methods. Conventional methods that can be employed include chromatography (such as HPLC) or supercritical fluid chromatography (SFC) on an asymmetric resin. Examples of useful resins include, but are not limited to, Chiralcel OJ-H, Chiralpak AD-H, Chiralpak IA and Chiralpak AS-H brand chiral stationary phases available from Daicel Chemical Industries, Ltd, Japan, with a mobile phase typically comprising an alcohol (e.g., from about 10% to about 50% by volume) and carbon dioxide. Concentration of the eluate affords the enriched mixture. The isomerically-enriched compounds may also be further derivatized.

The compounds of this invention may be prepared as described below, or suitable variation of the specific methods described below as will be well-known to those skilled in the art. In the reaction schemes and discussion that follow, Ar, X, Y, Z and R₁ are defined as above. Furthermore, Ar may be substituted with R₂ₐ, R_{2b} and R_{2c} as defined above.

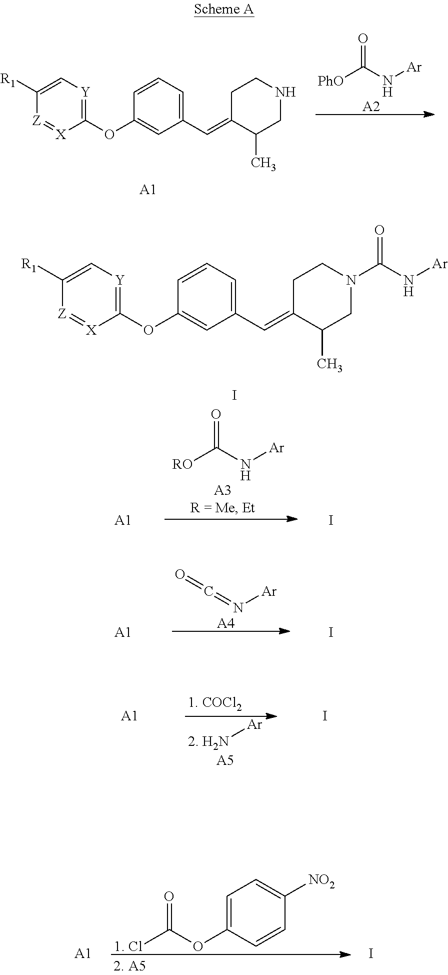

Compounds of Formula I can be prepared according to Scheme A. The reaction of a compound of formula A1 with a phenyl carbamate of formula A2 provides compounds of the Formula I. The reaction can be conducted in a polar aproptic solvent such as dimethylsulfoxide (DMSO) or acetonitrile. The temperature of the reaction may vary from about ambient temperature to about 60° C. The reaction may also be conducted using a trifluoroacetic acid or hydrochloride salt of the compound of formula A1 in the presence of a base such as triethylamine or diisopropylethyl amine. Alternatively, the reaction of a compound of formula A1 with a carbamate of formula A3 (R=Me or Et) under microwave irradiation may provide compounds of the Formula I. The reaction may be conducted in a solvent such as acetonitrile. The reaction may also be conducted using a trifluoroacetic acid or hydrochloride salt of the compound of formula A1 in the presence of a base such as triethylamine or diisopropylethyl amine. Furthermore, compounds of the Formula I may be prepared by reacting compounds of formula A1 with an isocyanate of formula A4. The reaction may be conducted in a solvent such as methylene chloride at ambient temperature. The reaction may also be conducted using a trifluoroacetic acid or hydrochloride salt of the compound of formula A1 in the presence of a base such as triethylamine or diisopropylethyl amine. Alternatively, compounds of formula A1 may be reacted with phosgene in the presence of a base such as triethylamine or diisopropylethylamine and a solvent such as dichloromethane at 0° C. to generate the chloroformate derivative of formula A1 which may be isolated as a crude material and reacted with amines of formula A5 in the presence of a base such as triethylamine or diisopropylethylamine and a catalyst such as 4-(dimethylamino)-pyridine in a suitable solvent such as acetonitrile, dichloromethane, and dichioroethane. The reaction temperature may vary from about ambient temperature to about 70° C. Alternatively, compounds of formula A1 may be reacted with 4-nitrophenyl chloroformate in the presence of a base such as aqueous sodium bicarbonate and a solvent such as dioxane at room temperature generate the 4-nitrophenyl carbamate derivative of formula A1 which may be isolated as a crude material, optionally purified, and reacted with amines of formula A5 in the presence of a base such as sodium hydride in a suitable solvent such as dimethylacetamide or dimethylformamide. The reaction temperature may vary from about ambient temperature to about 70° C.

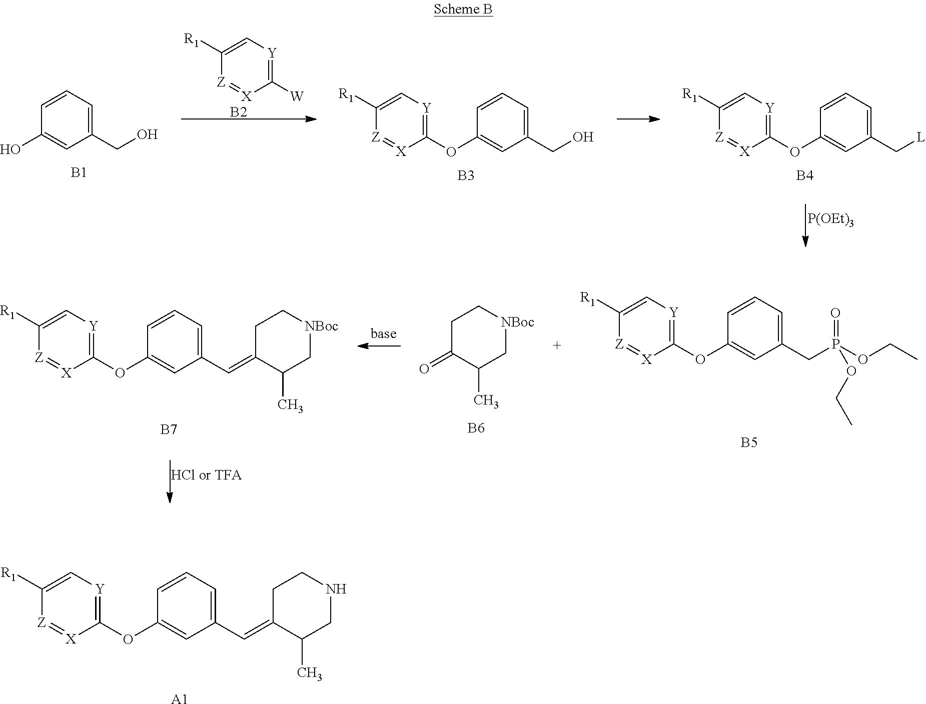

Compounds of formula A1 can be prepared according to Scheme B. The synthesis begins with a nucleophilic aromatic substitution of a phenol of formula B1 with an electron deficient aryl halide of the formula B2 (where X is N, Y is CH or N, Z is CH or N, and W is F or Cl) to form the biaryl ether of formula B3. This reaction is preferably run in the presence of a base such as potassium carbonate, sodium carbonate, cesium carbonate, triethylamine or diisopropylethylamine. The solvent used may be dimethylformamide (DMF), N-methylpyrrolidinone (NMP), dimethylsulfoxide (DMSO), acetonitrile, tetrahydrofuran, dioxane or a combination of two or more of these solvents. The hydroxy group of the compound of formula B3 is converted into a leaving group (L) using conventional methods (for example, using thionyl chloride) to provide the corresponding compound of formula B4 wherein L is a halogen such as bromide, iodide or chloride. The resulting compounds of formula B4 are then reacted with triethyl phosphite to give the corresponding phosphonates of formula B5. The reaction can be conducted neat or in a solvent such as toluene, xylene, or chlorobenzene. The temperature of the reaction may vary from about ambient temperature to about the reflux temperature of the solvent used. The reaction is preferably conducted with a compound of formula B4 where L=Cl or Br in refluxing triethyl phosphite. Horner-Wadsworth-Emmons olefination of a compound of formula B5 with 3-methyl-4-oxo-piperidine-1-carboxylic acid tert-butyl ester (B6) in the presence of a base provides the compound of formula B7. This reaction is conducted in the presence of a base such as potassium tert-butoxide, sodium tert-butoxide, sodium hydride, potassium hydride, lithium diisopropylamide, lithium bis(trimethylsilyl)amide, potassium bis(trimethylsilyl)amide, sodium bis(trimethylsilyl)amide, or butyllithium. The reaction can be conducted in a solvent such as tetrahydrofuran (THF), 2-methyltetrahydrofuran, dioxane, ethylene glycol dimethylether, dimethylformamide (DMF) or N-methylpyrrolidinone (NMP), and the temperature of the reaction may vary from about ambient temperature to about the reflux temperature of the solvent used. An additive such as 15-crown-5 can also be used to help promote the reaction. The compound of formula B7 is deprotected using conventional methods (for example, using HCl in dioxane, acetyl chloride in ethanol or trifluoroacetic acid in dichloromethane) to provide the corresponding compound of formula A1 which can be isolated as the free base or as the corresponding salt (hydrochloride or trifluoroacetate).

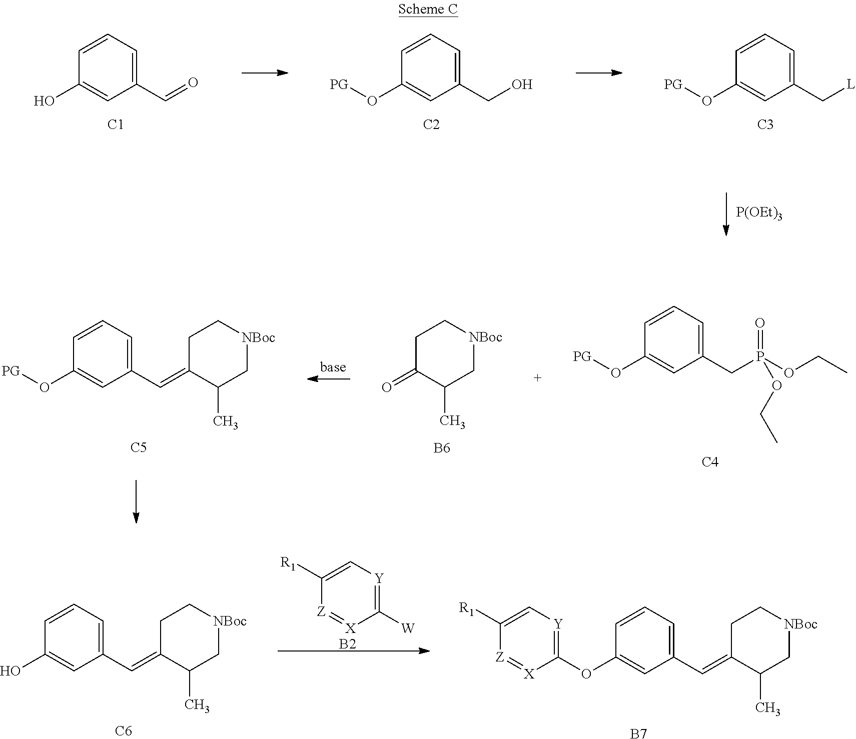

Scheme C illustrates a method for preparing compounds of formula B7. A phenolic aldehyde such as C1 may be protected with a protecting group (PG) such as tetrahydropyranyl (THP), benzyl (Bn), p-methoxybenzyl (PMB), tert-butyldimethysilyl (TBS), triisopropylsilyl (TIPS), tert-butyldiphenylsilyl (TBDPS) or pivaloyl (Pv) according to standard methods. The protected phenolic aldehyde may then be reduced to the alcohol of formula C2 with a reducing agent such as sodium borohydride. Compounds of formula C2 can then be converted into a leaving group (L) where L is Cl, Br or I, and then reacted with triethyl phosphite to give the phosphonate of formula 13 as described in Scheme B. Horner-Wadsworth-Emmons olefination of a compound of the formula 13 with 3-methyl-4-oxo-piperidine-1-carboxylic acid tert-butyl ester (B6) in the presence of a base provides the compound of formula C5 as described in Scheme B. Compounds of formula C5 wherein PG is tert-butyldimethysilyl, triisopropylsilyl (TIPS) or tert-butyldiphenylsilyl can be deprotected using conventional methods such as treatment with tetrabutylammonium fluoride in tetrahydrofuran to yield compounds of formula C6. Compounds of formula C5 where PG is tetrahydropyranyl (THP) can be deprotected using conventional methods such as treatment with PPTS (pyridinium p-tolunesulfonate) or p-toluenesulfonic acid in ethanol to give the corresponding compounds of formula C6. Compounds of formula C5 where PG is pivaloyl (Pv) can be deprotected using conventional methods such as treatment with lithium aluminium hydride in THF to give the corresponding compounds of formula C6. Nucleophilic aromatic substitution of a phenol of the formula C6 with an electron deficient aryl halide of the formula B2 (where X is N, Y is CH or N, Z is CH or N, and W is F or Cl) provides the biaryl ether of formula B7 as described in Scheme B.

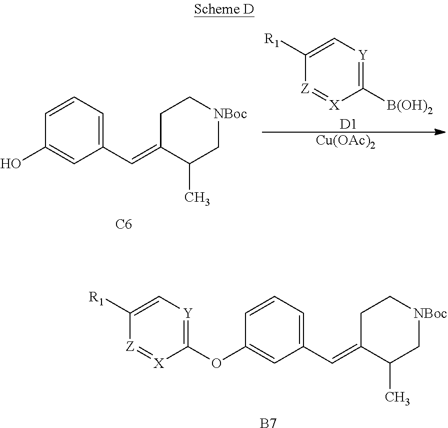

Scheme D illustrates another method for preparing compounds of Formula B7. Copper(II)-promoted coupling of a phenol of formula C6 with a boronic acid of the formula ArB(OH)₂ (D1; where X and Y are CH and Z is CH or N) may provide the biaryl ether of formula B7. The reaction may be conducted with one (1) equivalent of copper(II)acetate and 5-10 equivalents of triethylamine in a solvent such as methylene chloride with 4 Å molecular sieves at ambient temperature (Tetrahedron Let. 1998, 39, 2937).

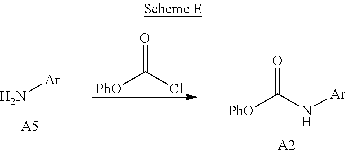

Scheme E illustrates a method for making phenyl carbamates of formula A2. Treatment of an aryl amine of formula A5 with phenyl chloroformate in a solvent such as THF, methylene chloride, or 1,4-dioxane gives phenyl carbamates of formula A2 in a manner similar to that described in Synthesis, 1997, 1189-1194. The reaction may be performed in the presence of a base such as triethylamine, diisopropylethylamine, and the like. The temperature of the reaction may vary from about 0° C. to reflux temperature of the solvent being used.

### EXAMPLES

The following examples are intended to illustrate particular embodiments of the invention and are not intended to limit the scope of the claims.

¹H Nuclear magnetic resonance (NMR) spectra were obtained for the compounds in the following examples. Characteristic chemical shifts (δ) are given in parts-per-million downfield from tetramethylsilane using conventional abbreviations for designation of major peaks, including s (singlet), d (doublet), t (triplet), q (quartet); m (multiplet), and br (broad). The mass spectra were recorded using electrospray (ES) or atmospheric pressure chemical ionization (APCI). The following abbreviations are used for common solvents: CDCl₃ (deuterochloroform), DMSO-d₆ (deuterodimethylsulfoxide).

### Synthesis of[3-(5-Trifluoromethyl-pyridin-2-yloxy)-phenyl]-methanol

3-Hydroxybenzyl alcohol (5.00 g, 40.3 mmol; CAS# 620-24-6), 2-chloro-5-trifluoromethyl-pyridine (7.31 g, 40.3 mmol; CAS# 52334-81-3) and potassium carbonate (6.96 g, 50.3 mmol) were suspended in DMF (80 mL) and heated to 95 ° C. After stirring for 16 h, the solvent was distilled off in vacuo at 65 ° C., and a residue was partitioned between water and heptane/ethyl acetate (1:1). The organic layer was separated and the aqueous was extracted again with heptane/ethyl acetate (1:1). The combined organic layer was dried over sodium sulfate, filtered and concentrated to give a residue. The residue was purified by silica gel chromatography (10-60%, EtOAc:heptane) to afford the desired product (5.70 g, 53% yield) as a light yellow oil. ¹H NMR (400 MHz, CDCl₃) δ ppm 4.73 (s, 2 H) 7.02 (dt, J=8.66, 0.57 Hz, 1 H) 7.04-7.11 (m, J=8.06, 2.40, 0.50, 0.50 Hz, 1 H) 7.15-7.19 (m, 1 H) 7.25 (ddd, J=8.39, 1.60, 0.80 Hz, 1 H) 7.42 (t, J=7.87 Hz, 1 H) 7.90 (ddd, J=8.67, 2.55, 0.50 Hz, 1 H) 8.43 (td, J=1.68, 0.84 Hz, 1 H).

### Synthesis of 2-(3-Chloromethyl-phenoxy)-5-trifluoromethyl-pyridine

[3-(5-Trifluoromethyl-pyridin-2-yloxy)-phenyl]-methanol (4.68 g, 17.4 mmol), in dichloromethane (46 mL), was cooled to 0° C., and treated dropwise with thionyl chloride (1.40 mL, 19.1 mmol). The reaction mixture was allowed to warm to ambient temperature and was stirred for 30 min. Toluene (10 mL) was added and the mixture was concentrated by evaporation to form a residue. The residue was evaporated again from toluene and dried under high vacuum to afford the desired product (4.88 g, 98% yield) as an oil. ¹H NMR (400 MHz, CDCl₃) δ ppm 4.60 (s, 2 H) 7.03 (d, J=8.70 Hz, 1 H) 7.11 (ddd, J=8.09, 2.35, 0.94 Hz, 1 H) 7.20 (t, J=2.03 Hz, 1 H) 7.26-7.31 (m, 1 H) 7.42 (t, J=7.88 Hz, 1 H) 7.91 (dd, J=8.67, 2.53 Hz, 1 H) 8.44 (dd, J=1.51, 0.90 Hz, 1 H).

### Synthesis of [3-(5-Trifluoromethyl-pyridin-2-yloxy)-benzyl]-phosphonic acid diethyl ester

2-(3-Chloromethyl-phenoxy)-5-trifluoromethyl-pyridine (4.88 g, 17.0 mmol) was treated neat with triethylphosphite (4.36 mL, 25.4 mmol) and heated to 150° C. After 6 h, the reaction mixture was cooled, treated with an additional 0.5 mL triethylphosphite (2.9 mmol) and reheated to 150° C. After 6 h, the reaction mixture was removed from the heat and slowly treated with heptane (about 60 mL) while stirring to afford a white solid. The solid was collected by filtration, washed with heptane and dried in a vacuum oven for 16 h at 45° C. to afford a white powder (5.99 g, 91% yield). MS (APCI) M+1=390.1; ¹H NMR (400 MHz, CDCl₃) δ ppm 1.26 (t, J=7.02 Hz, 6 H) 3.18 (d, J=21.83 Hz, 2 H) 3.99-4.10 (m, 4 H) 7.01 (d, J=8.58 Hz, 1 H) 7.03-7.08 (m, 1 H) 7.12 (q, J=2.21 Hz, 1 H) 7.19-7.24 (m, 1 H) 7.38 (t, J=7.90 Hz, 1 H) 7.90 (dd, J=8.58, 2.53 Hz, 1 H) 8.43 (dd, J=1.66, 0.88 Hz, 1 H).

### Synthesis of 2-{3-[(E)-(3-metylpiperidin-4-ylidene)methyl]phenoxy}-5-(trifluoromethyl)pyridine

[3-(5-Trifluoromethyl-pyridin-2-yloxy)-benzyl]-phosphonic acid diethyl ester (8.05 g, 20.7 mmol) and tert-butyl 3-methyl-4-oxopiperidine-1-carboxylate (4.20 g, 19.69 mmol; CAS# 181269-69-2) were dissolved in THF (50 mL) and t-BuOK (3.31 g, 29.50 mmol) was added. The solution was stirred at r.t. for 1 hr. at which time it was quenched with water (10 mL) and extracted with ethyl acetate (3×20 mL). The organics were dried with sodium sulfate and concentrated to give the olefin as a clear oil. The oil was dissolved in dichloromethane (20 mL) and trifluoroacetic acid (5.0 mL) added at r.t. and the solution stirred for 1 hr. The reaction was then neutralized with sodium bicarbonate (20 mL) and extracted with dichloromethane (2×50 mL). The organics were dried with sodium sulfate and concentrated. The crude product was purified by flash chromatography (35% ethyl acetate/ heptane) to produce the title compound as an oil (racemic mixture of E and Z isomers, 5.42 g, 15.57 mmol, 79%).

### Synthesis of 3-formylphenyl pivalate

3-hydroxybenzaldehyde (15.00 g, 122.83 mmol; CAS# 100-83-4), was dissolved in THF (150 mL) and pyridine (9.93 mL, 123.0 mmol) added followed by pivaloyl chloride (15.10 mL, 123.0 mmol) The reaction was stirred at r.t. for 1 hour at which time it was quenched with water and the aqueous phase extracted with dichloromethane (2×100 mL). The organics were dried with magnesium sulfate and concentrated. The crude product was purified by flash chromatography (20% ethyl acetate/heptane) to produce the title compound as an oil (23.65 g, 114.67 mmol, 93%).

### Synthesis of 3-(bromomethyl)phenyl pivalate

3-formylphenyl pivalate (25.0 g, 121.22 mmol) was dissolved in MeOH (100mL) and sodium borohydride (5.56 g, 145.0 mmol) added portionwise at r.t. The reaction was stirred for an additional 2 hours at which time it was quenched with water and the aqueous phase extracted with dichloromethane (2×100 mL). The organics were dried with magnesium sulfate and concentrated to produce crude 3-(hydroxymethyl)phenyl pivalate. The crude 3-(hydroxymethyl)phenyl pivalate (13.0 g, 62.42 mmol) was then dissolved in dichloromethane (65 mL) and imidazole (4.55 g, 65.50 mmol) added followed by triphenylphosphine (18.60 g, 68.70 mmol) and bromine (3.21 mL, 62.4 mmol) at 0° C. The reaction was warmed to r.t. and stirred for 30 minutes at which time it was quenched with water and the aqueous phase extracted with dichloromethane (2×50 mL). The organics were dried with magnesium sulfate and concentrated. The crude product was purified by flash chromatography (20% ethyl acetate/heptane) to produce the title compound as an oil (14.00 g, 51.66 mmol, 83%). m/z 270.9(MH⁺).

### Synthesis of tert-butyl-4-{3-[(2,2-dimethylpropanoyl)oxy]benzylidene}-3-methylpiperidine-1-carboxvlate

3-(bromomethyl)phenyl pivalate (2.0g, 7.38 mmol) and triethyl phosphite (1.28 mL, 7.38 mmol) were heated at 150° C. for 4 hours. The solution was then cooled and the excess triethyl phosphite vacuumed off to give crude 3-[(diethoxyphosphoryl)methyl]phenyl pivalate. The crude product was dissolved in THF (21 mL) and tert-butyl 3-methyl-4-oxopiperidine-1-carboxylate (1.57 g, 7.38 mmol; CAS# 181269-69-2) followed by potassium t-butoxide (828 mg, 7.38 mmol) added. The reaction was stirred at r.t. for 2 hours and quenched with water. The aqeous phase was extracted with ethyl acetate(2×25 mL) dried with magnesium sulfate and concentrated. The crude product was purified by flash chromatography (25% ethyl acetate/heptane) to produce the title compound as an oil (980 mg, 2.62 mmol, 36%).

### Synthesis of tert-butyl 4-(3-hydroxybenzylidene)-3-methylpiperidine-1-carboxylate

tert-butyl 4-{3-[(2,2-dimethylpropanoyl)oxy]benzylidene}-3-methylpiperidine-1-carboxylate (1.65 g, 4.26 mmol) was dissolved in THF (12 mL) and lithium aluminium hydride (4.26 mL, 4.26 mmol) added at r.t.

The reaction was stirred overnight and quenched with water. The product was extracted with ethyl acetate (2×25 mL), dried with magnesium sulfate and concentrated. The crude product was purified by flash chromatography (35% ethyl acetate/heptane) to produce the title compound as an oil (920 mg, 3.02 mmol, 71%).

### Synthesis of 2-{3-[3-methylpiperidin-4-ylidenemethyl]phenoxy}-5-(trifluoromethyl)pyrimidine

tert-butyl 4-(3-hydroxybenzylidene)-3-methylpiperidine-1-carboxylate (990 mg, 3.20 mmol) was dissolved in DMSO (6 mL) and 5-(trifluoromethyl)pyrimidine (595.6 mg, 3.26 mmol; CAS# 69034-12-4) followed by Cs₂CO₃ (1.68 g, 4.89 mmol) was added. The solution was heated to 65° C. for 1 hr at which time it was cooled to r.t. The solution was quenched with water (10 mL) and extracted with ethyl acetate (25 mL). The organics were dried with sodium sulfate and concentrated. The crude product was vacuum filtrated through a silica plug (10% ethyl acetate/heptane) to produce the crude biaryl ether as a pale yellow oil. The oil was dissolved in dichloromethane (20 mL) and trifluoroacetic acid (5.0 mL) added at r.t. and the solution stirred for 1 hr. The reaction was then neutralized with sodium bicarbonate (20 mL) and extracted with dichloromethane (2×50 mL). The organics were dried with sodium sulfate and concentrated. The crude product was purified by flash chromatography (60% ethyl acetate/heptane) to produce the title compound as an oil (racemic mixture of E and Z isomers, 790 mg, 2.26 mmol, 70%)

### Synthesis of 3-(5-Bromopyridin-2-yloxy)phenyl)methanol

3-Hydroxymethyl-phenol (3.205 g, 25,82 mmol), 5-bromo-2-fluoropyridine (5.00 g, 28.4 mmol; CAS# 766-11-0) and cesium carbonate (9.26 g, 28.4 mmol) were suspended in DMSO (40 mL) and heated to 100° C. After stirring for 16 h, the reaction mixture was partitioned between water (400 mL) and ethyl acetate (400 mL). The organic layer was separated and the aqueous layer was extracted again with ethyl acetate. The combined organic layers were dried over sodium sulfate, filtered and concentrated. The residue was purified by flash chromatography on silica (10-60%, EtOAc:heptane) to afford the desired product (5.71 g, 79% yield) as a clear oil. MS (APCI) M+1=280.0; ¹H NMR (400 MHz, CDCl₃) δ ppm 1.82 (s, 1 H) 4.70 (s, 2 H) 6.84 (dd, J=8.58, 0.58 Hz, 1 H) 7.00-7.06 (m, 1 H) 7.13 (t, J=1.75 Hz, 1 H) 7.17-7.23 (m, 1 H) 7.38 (t, J=7.80 Hz, 1 H) 7.76 (dd, J=8.77, 2.53 Hz, 1 H) 8.20 (dd, J=2.63, 0.49 Hz, 1 H).

### Synthesis of 5-Bromo-2-(3-(chloromethyl)phenoxy)pyridine

3-(5-Bromopyridin-2-yloxy)phenyl)methanol (3.00 g, 10.7 mmol), in dichloromethane (30 mL), was cooled to 0° C., and treated dropwise with thionyl chloride (0.86 mL, 11.8 mmol). The reaction mixture was allowed to warm to ambient temperature and was stirred for 1 h. Toluene (5 mL) was added and the mixture was concentrated by evaporation. The residue was evaporated again from toluene and dried under high vacuum to afford the desired product (3.09 g, 97% yield) as a white semi-solid. MS (APCI) M+1=300.0; ¹H NMR (400 MHz, CD₃OD) δ ppm 4.64 (s, 2 H) 6.93 (dd, J=8.68, 0.68 Hz, 1 H) 7.07 (ddd, J=8.19, 1.17, 0.97 Hz, 1 H) 7.15-7.21 (m, 1 H) 7.24-7.30 (m, 1 H) 7.40 (t, J=7.90 Hz, 1 H) 7.94 (dd, J=8.77, 2.53 Hz, 1 H) 8.19 (dd, J=2.53, 0.58 Hz, 1 H).

### Synthesis of Diethyl 3-(5-bromopyridin-2-yloxy)benzylphosphonate

5-Bromo-2-(3-(chloromethyl)phenoxy)pyridine (3.08 g, 10.3 mmol) from Step 2 was treated neat with triethylphosphite (2.65 mL, 15.5 mmol) and heated to 150° C. After 5 h, the reaction mixture was removed from the heating bath and treated slowly with heptane until an oil precipitated out of solution. Ethyl acetate was added until mixture became homogenous. Heptane was slowly added again (at a cooler temperature) until a white precipitate formed. After the addition of more heptane and stirring for 15 min, the precipitate was filtered to afford a white solid (3.35 g, 81% yield). MS (APCI) M+1=400.0; ¹H NMR (400 MHz, CDCl₃) δ ppm 1.26 (t, J=7.02 Hz, 6 H) 3.17 (d, J=21.64 Hz, 2 H) 3.98-4.08 (m, 4 H) 6.84 (d, J=8.77 Hz, 1 H) 7.00-7.05 (m, 1 H) 7.08 (q, J=2.21 Hz, 1 H) 7.15-7.20 (m, 1 H) 7.35 (t, J=7.90 Hz, 1 H) 7.77 (dd, J=8.58, 2.53 Hz, 1 H) 8.21 (d, J=2.73 Hz, 1 H).

### Synthesis of 5-bromo-2-{3-[(3-methylpiperidin-4-ylidene)methyl]phenoxy}pyridine

Diethyl 3-(5-bromopyridin-2-yloxy)benzylphosphonate (7.50 g, 18.74 mmol) was dissolved in THF (50 mL) and tert-butyl 3-methyl-4-oxopiperidine-1-carboxylate (5.46 g, 25.6 mmol) followed by potassium t-butoxide (2.39 g, 21.30 mmol) added. The reaction was stirred at r.t. for 2 hours and quenched with water. The aqeous phase was extracted with ethyl acetate(2×50 mL) dried with magnesium sulfate and concentrated give the olefin as a clear oil. The oil was dissolved in dichloromethane (20 mL) and trifluoroacetic acid (5.0 mL) added at r.t. and the solution stirred for 1 hr. The reaction was then neutralized with sodium bicarbonate (20 mL) and extracted with dichloromethane (2×50 mL). The organics were dried with sodium sulfate and concentrated. The crude product was purified by flash chromatography (60% ethyl acetate/heptane) to produce the title compound as an oil (racemic mixture of E and Z isomers, 5.50 g, 15.34 mmol, 72%).

### Synthesis of 3-[(5-chloropyridin-2-yl)oxy]benzaldehyde

3-hydroxybenzaldehyde (5.0 g, 33.79 mmol), 2,5-dichloropyridine (3.92 g, 32.1 mmol; CAS# 16110-09-1) and cesium carbonate (11.6 g, 33.8 mmol) were suspended in DMSO (40 mL) and heated to 100° C. After stirring for 16 h, the reaction mixture was partitioned between water (400 mL) and ethyl acetate (400 mL). The organic layer was separated and the aqueous layer was extracted again with ethyl acetate. The combined organic layers were dried over sodium sulfate, filtered and concentrated. The residue was purified by flash chromatography on silica (25%, EtOAc:heptane) to afford the desired product (4.55 g, 19.53 mmol, 58% yield) as a clear oil.

### Synthesis of 2-[3-(bromomethyl)phenoxy]-5-chloropyridine

3-[(5-chloropyridin-2-yl)oxy]benzaldehyde (4.55 g, 19.47 mmol) was dissolved in MeOH (100mL) and sodium borohydride (1.12 g, 29.2 mmol) added portionwise at r.t. The reaction was stirred for an additional 2 hours at which time it was quenched with water and the aqueous phase extracted with dichloromethane (2×50 mL). The organics were dried with magnesium sulfate and concentrated to produce crude {3-[(5-chloropyridin-2-yl)oxy]phenyl}methanol. The crude {3-[(5-chloropyridin-2-yl)oxy]phenyl}methanol (4.50 g, 19.09 mmol) was then dissolved in dichloromethane (40 mL) and imidazole (1.39 g, 20.00 mmol) added followed by triphenylphosphine (5.68 g, 21.00 mmol) and bromine (0.98 mL, 19.1 mmol) at 0° C. The reaction was warmed to r.t. and stirred for 30 minutes at which time it was quenched with water and the aqueous phase extracted with dichloromethane (2×50 mL). The organics were dried with magnesium sulfate and concentrated. The crude product was purified by flash chromatography (20% ethyl acetate/heptane) to produce the title compound as an oil (4.96 g, 16.64 mmol, 87%).

### Synthesis of 5-chloro-2-{3-[(E)-(3-methylpiperidin-4-ylidene)methyl]phenoxy}pyridine

2-[3-(bromomethyl)phenoxy]-5-chloropyridine (4.96g, 16.61 mmol) and triethyl phosphite (2.89 mL, 16.60 mmol) were heated at 150 ° C. for 4 hours. The solution was then cooled and the excess triethyl phosphite vacuumed off to give crude diethyl {3-[(5-chloropyridin-2-yl)oxy]benzyl}phosphonate. The crude product was dissolved in THF (21 mL) and tert-butyl 3-methyl-4-oxopiperidine-1-carboxylate (4.25 g, 19.9 mmol; CAS# 181269-69-2) followed by potassium t-butoxide (2.80 g, 24.9 mmol) added. The reaction was stirred at r.t. for 2 hours and quenched with water. The aqeous phase was extracted with ethyl acetate(2×25 mL) dried with magnesium sulfate and concentrated give the olefin as a clear oil. The oil was dissolved in dichloromethane (20 mL) and trifluoroacetic acid (5.0 mL) added at r.t. and the solution stirred for 1 hr. The reaction was then neutralized with sodium bicarbonate (20 mL) and extracted with dichloromethane (2×50 mL). The organics were dried with sodium sulfate and concentrated. The crude product was purified by flash chromatography (60% ethyl acetate/ heptane) to produce the title compound as an oil (racemic mixture of E and Z isomers, 4.71 g, 14.94 mmol, 90%).

### Synthesis of Phenyl pyridin-3-ylcarbamate

To a stirred solution of 3-aminopyridine (51.7 g, 0.549 moles; CAS# 462-08-8) in THF (900 mL) at −10° C. was added pyridine (52.1 g, 0.659 moles) in a stream over a 10 min period, followed by the dropwise addition of phenyl chloroformate (90 g, 0.575 moles) over a 20 min period. The reaction tempature increased to 5° C. A precipitate formed during the addition. The resulting suspension was stirred at temperatures reaching ambient temperature over the next 3 h. The reaction mixture was partitioned between water (2 L) and EtOAc (1.5 L). The aqueous portion was extracted with EtOAc (1 L). The combined organic portions were dried (MgSO₄) and concentrated in vacuo to a damp solid residue. This was suspended in EtOAc:ether (1:1, 600 mL). The resulting suspension was stirred at −10° C. for 2 h and filtered. The solid was rinsed with EtOAc:ether (1:1, 100 mL) and pressed dry under suction. Further drying in vacuo at 35° C. for 7 h provided 104 g (88%) of product. Analysis, Calcd for C₁₂H₁₀N₂O₂: C, 67.28; H, 4.71; N, 13.08. Found: C, 67.15; H, 4.76; N, 12.87.

### Synthesis of Phenyl pyridazin-3-ylcarbamate

To a solution of 3-amino-6-chloropyridazine (19.2 g,148 mmol; CAS# 5469-69-2) in EtOH (500 mL) was added 10% Pd catalyst on 1940 carbon (unreduced, 55% water). Triethylamine (50 mL) was added and the mixture was hydrogenated under 500 psi/mole for 1.9 h. The reaction was filtered and the ethanol was washed with aqueous NH₄Cl. The organic layer was concentrated to give pyridazin-3-amine as a white solid (11 g, 78% yield). MS (APCI 10V) AP+1 96.2. To a suspension of pyridazin-3-amine (5 g, 50 mmol) in THF (50 mL) and CH₃CN (70 mL) was added pyridine (5.10 mL, 63.1 mmol) followed by phenyl chloroformate (6.95 mL, 55.2 mmol) slowly. The reaction was stirred overnight. The reaction was filtered to remove the precipitate. The filtrate was concentrated and then taken up in CH₂Cl₂ which was washed with water. The organic layer was dried using SPE phase separators and concentrated. The residue was purified by silica gel column chromatography (0-5% MeOH/CH₂Cl₂). An undesired side product eluted first followed by the title compound which was concentrated to give a white solid (7.5 g, 70% yield). MS (APCI 10V) AP+1 216.12; ¹H NMR (400 MHz, DMSO-d₆) δ ppm 7.20-7.24 (m, 2 H) 7.25-7.28 (m, 1 H) 7.39-7.44 (m, 2 H) 7.64-7.69 (m, 1 H) 8.05 (dd, 1 H) 8.94 (dd, 1 H) 11.34 (s, 1 H).

### Synthesis of phenyl (3,4-dimethylisoxazol-5-yl)carbamate

To a solution of 5-amino-3,4-dimethylisoxazole (2.00 g, 17.8 mmol, 1.0 equiv; CAS# 19947-75-2) in THF (180 mL) at 0° C. was added pyridine (1.80 mL, 22.3 mmol, 1.25 equiv) followed by phenyl chloroformate (2.36 mL, 18.7 mmol, 1.05 equiv). After stirring at 0° C. for 2.5 h, the reaction was warmed to room temp overnight. The reaction was diluted with ethyl acetate and washed with 2M HCl, water, saturated sodium bicarbonate, and brine. The organic layer was dried over magnesium sulfate, filtered, concentrated, and purified by flash chromatography (dichloromethane/hexane) to give the title compound as a white solid (2.33 g). ¹H NMR (400 MHz, DMSO-d₆) δ ppm 10.70 (br. s., 1 H), 7.40-7.47 (m, 2 H), 7.26-7.30 (m, 1 H), 7.21-7.25 (m, 2 H), 2.16 (s, 3 H), 1.86 (s, 3 H). m/z 233 (MH⁺).

### Synthesis of phenyl (5-methylisoxazol-4-yl)carbamate

To a solution of 4-amino-5-methylisoxazole (2.00 g, 20.39 mmol; CAS# 87988-94-1) in THF (50 mL) at 0° C. was added pyridine (1.65 mL, 20.39 mmol) followed by phenyl chloroformate (2.81 mL, 22.43 mmol). After stirring at 0° C. for 2.5 h, the reaction was warmed to room temp overnight. The reaction was diluted with ethyl acetate and washed with 2M HCl, water, saturated sodium bicarbonate, and brine. The organic layer was dried over magnesium sulfate, filtered, concentrated, and purified by flash chromatography (ethyl acetate (5% ethanol)/ heptanes) to give the title compound as a white solid (2.85 g, 13.07 mmol, 64%).

### Synthesis of phenyl isoxazol-4-ylcarbamate

To a solution of 4-aminoisoxazole (2.00 g, 23.79 mmol; CAS# 108511-97-3) in THF (50 mL) at 0° C. was added pyridine (1.92 mL, 23.79 mmol,) followed by phenyl chloroformate (3.28 mL, 26.17 mmol,). After stirring at 0° C. for 2.5 h, the reaction was warmed to room temp overnight. The reaction was diluted with ethyl acetate and washed with 2M HCl, water, saturated sodium bicarbonate, and brine. The organic layer was dried over magnesium sulfate, filtered, concentrated, and purified by flash chromatography (dichloromethane/hexane) to give the title compound as a white solid (2.07 g, 10.15 mmol, 50%).

### Synthesis of phenyl (6-methoxypyridin-3-yl)carbamate

3-Amino-6-methoxypyridine (5.00 g, 40.3 mmol; CAS# 6628-77-9) was dissolved in THF (80 mL), cooled to 0° C., treated with pyridine (4.07 mL, 50.4 mmol) followed by phenylchloroformate (5.32 mL, 42.3 mmol). The reaction mixture was slowly warmed to RT over several hours and stirred an additional 12 h. The mixture was partitioned between water and EtOAc. The organic layer was separated and the aqueous layer was extracted again. The combined organic extracts were dried over sodium sulfate, filtered and concentrated to give the product as a reddish solid (9.45 g, 96%), which was used without purification.

### Synthesis of phenyl 1H-pyrrolo[2,3-b]pyridin-5-ylcarbamate

To a solution of 1 H-pyrrolo[2,3-b]pyridin-5-ylamine (0.50 g, 3.7 mmol; Synthesis, 2005, 2503-2506; CAS# 100960-07-4) in THF (4 mL) and CH₃CN (6 mL) was added pyridine (0.36 mL, 4.4 mmol) followed by phenyl chloroformate (0.49 mL, 3.8 mmol) slowly. The reaction was stirred overnight. The mixture was partitioned between water and EtOAc. The aqueous layer was extracted again with EtOAc. The combined organic layer was dried over sodium sulfate, filtered and concentrated. Purification by chromatography (0-100% EtOAc/hexane) provided the desired product as a white solid (0.213 g, 23%). m/z 254.15 (MH⁺).

### Synthesis of phenyl (5-methylpyridin-3-yl)carbamate

A solution of 3-amino-5-methylpyridine (2.00 g, 18.5 mmol; CAS# 3430-19-1) in THF (100 mL) at 0 ° C. was treated with pyridine (1.8 mL, 22.2 mmol) followed by a solution of phenyl chloroformate (2.8 mL, 22.2 mmol) in THF (20 mL) dropwise. After 3 h, the reaction was diluted with water (100 mL) and ethyl acetate (250 mL). The organic layer was washed with brine, dried over sodium sulphate, filtered and concentrated to ˜50 mL. The resulting white solid was filtered and rinsed with diethyl ether to give the title compound as a white solid (3.00 g, 71% yield).

### Synthesis of phenyl (6-cyanopyridin-3-yl)carbamate

A solution of 5-amino-2-cyanopyridine (2.00 g, 16.7 mmol; CAS# 55338-73-3) in THF (100 mL) at 0° C. was treated with pyridine (1.4 mL, 16.8 mmol) followed by a solution of phenyl chloroformate (2.1 mL, 16.8 mmol) in THF (20 mL) dropwise. After 3 h, the reaction was diluted with water (100 mL) and ethyl acetate (250 mL). The organic layer was washed with brine, dried over sodium sulphate, filtered and concentrated to ˜50 mL. The resulting beige solid was filtered to give the title compound (3.4 g, 85% yield).

### Synthesis of phenyl (5-methoxypyrazin-2-yl)carbamate

2-Amino-5-methoxypyrazine (1.5 g, 12.0 mmol; CAS# 54013-07-9) was dissolved in anhydrous THF under nitrogen and cooled in a dry ice-acetone bath. To this solution was added pyridine (2.3 mL, 28.8 mmol). After 30 min phenyl chloroformate (1.7 mL, 13.2 mmol) was added dropwise. The reaction mixture was allowed to gradually warm to room temperature. After 2 hours ethyl acetate (90 mL) and water (45 mL) were added. The aqueous layer was back extracted once with ethyl acetate. The combined organic layers were washed twice with brine, then dried (MgSO₄), filtered and concentrated. The residue was triturated with ether: ethyl acetate (50 mL: 2 mL). The solid was collected by vacuum filtration and washed with ether. To give the title compound as a pale yellow solid (2.5 g, 10.1 mmol, 85%) ¹H NMR (400 MHz, DMSO-d₆) δ ppm 10.79 (s, 1H), 8.58 (s, 1H), 8.13 (s, 1H), 7.36-7.52 (m, 2H), 7.16-7.34 (m, 3H), 3.88 (s, 3H). m/z 246.1

### Synthesis of phenyl (5-methylpyrazin-2-yl)carbamate

2-Amino-5-methyl-pyrazine (2.00 g, 21.25 mmol; CAS# 5521-58-4) was dissolved in THF (80 mL), cooled to 0° C., and treated with pyridine (1.77 g, 22.3 mmol) followed by the dropwise addition of phenylchloroformate (3.49 g, 22.3 mmol) in THF (30 mL). After stirring for 3 h, 100 mL of MeCN was added and the reaction mixture was reduced to a volume of 100 mL in vacuo. The title compound as white crystals was collected by filtration (2.5 g, 55%) and was used without further purification.

### Synthesis of phenyl (6-methoxypyrazin-2-yl)carbamate

2-Amino-6-methoxy-pyrazine (1.00 g, 8 mmol; CAS# 6905-47-1) was dissolved in a mix of 1:2 THF:MeCN (30 mL), cooled to 0° C., and treated with pyridine (0.664 g, 8.3 mmol) followed by the dropwise addition of phenylchloroformate (1.3 g, 8.3 mmol) in THF (10 mL). After stirring for 18 h, the resulting white solid was collected by filtration (1.3 g, 68%) and was used without further purification.

### Synthesis of phenyl (6-methoxypyridazin-3-yl)carbamate

A solution of 3-amino-6-methoxypyridazine (1.25 g, 10.0 mmol; CAS# 7252-84-8) in a mix of 1:1 THF:MeCN (20 mL), and pyridine (0.83 g, 1.5 mmol) was treated dropwise with phenylchloroformate (1.65 g, 10.5 mmol) in THF (10 mL). After stirring for 3 h, the resulting solid was collected and dried to provide the title compound (2 g, 81%).

### Synthesis of phenyl 1,2-benzisoxazol-3-ylcarbamate

A solution of 1,2-benzisoxazol-3-amine (1.00 g; CAS# 36216-80-5) and triethylamine (1.09 mL) in acetonitrile (5 mL) was added dropwise to at 0° C. solution of phenyl chloroformate (0.989 mL) in THF (20 mL). The reaction was stirred at 0° C. for 1 h and then allowed to warm to room temp overnight. The reaction was diluted with ethyl acetate and washed with 1N HCl and brine. The organic layer was dried over sodium sulfate, filtered, and concentrated to give the crude product as a reddish brown solid. The solid was triturated with refluxing diisopropyl ether, cooled to room temp, and filtered to give the final product as a tan solid (1.22 g, 64%). m/z 255 (MH⁺).

### Synthesis of Phenyl pyridin-3-ylcarbamate

To a stirred solution of 3-aminopyridine (51.7 g, 0.549 moles; CAS# 462-08-8) in THF (900 mL) at −10° C. was added pyridine (52.1 g, 0.659 moles) in a stream over a 10 min period, follovved by the dropwise addition of phenyl chloroformate (90 g, 0.575 moles) over a 20 min period. The reaction tempature increased to 5° C. A precipitate formed during the addition. The resulting suspension was stirred at temperatures reaching ambient temperature over the next 3 h. The reaction mixture was partitioned between water (2 L) and EtOAc (1.5 L). The aqueous portion was extracted with EtOAc (1 L). The combined organic portions were dried (MgSO4) and concentrated in vacuo to a damp solid residue. This was suspended in EtOAc:ether (1:1, 600 mL). The resulting suspension was stirred at −10° C. for 2 h and filtered. The solid was rinsed with EtOAc:ether (1:1, 100 mL) and pressed dry under suction. Further drying in vacuo at 35° C. for 7 h provided 104 g (88%) of product. Analysis, Calcd for C₁₂H₁₀N₂O₂: C, 67.28; H, 4.71; N, 13.08. Found: C, 67.15; H, 4.76; N, 12.87.

### Synthesis of (4E)-3-methyl-N-pyridin-3-yl-4-(3-{{5-(trifluoromethyl)pyridin-2-yl]oxy]benzylidene)piperidine-1-carboxamide

A mixture of 2-{3-[(3-methylpiperidin-4-ylidene)methyl]phenoxy)-5-(trifluoromethl)pyridine (5.60 g, 16.08 mmol) and phenyl pyridin-3-ylcarbamate (3.44 g, 16.10 mmol) in acetonitrile (25 mL) was treated with diisopropylamine (2.86 mL, 16.1 mmol) and stirred for 1 hr at r.t. The reaction was concentrated to give the crude product as a racemic mixture of olefin isomers. The isomers were separated by chiral SFC (Chiralpak AS-H column (20% n-butanol/CO₂), followed by Chiralpak AD-H (20% MeOH/CO₂) and Chiralpak AS-H (20% EtOH/CO₂)) to give the two enantiomers of the E-olefin. (4E)-3-methyl-N-pyridin-3-yl-4-(3-{{5-(trifluoromethyl)pyridin-2-yl]oxy}benzylidene)piperidine-1-carboxamide (enantiomer 1). Second eluting peak, t_{R}=1.93 min (4.6×150 mm Chiralpak AS-H, 20% ethanol/CO₂ at 3 mL/min). ¹H NMR (400 MHz, DMSO-d₆) δ ppm 8.75 (1 H, s), 8.64 (1 H, d, J=2.4 Hz), 8.57 (1 H, s), 8.23 (1 H,dd, J=8.7, 2.5 Hz), 8.14 (1 H, dd, J=4.7, 1.2 Hz), 7.84-7.92 (1 H, m), 7.42 (1 H, t, J=7 .7 Hz), 7.28 (1 H,dd, J=8.3, 4.7 Hz), 7.23 (1 H, d, J=8.8 Hz), 7.14 (1 H, d, J=7.7 Hz), 7.01-7.09 (2 H, m), 6.36 (1 H, s),3.80 (1 H, dd, J=12.9, 3.9 Hz), 3.61-3.74 (1 H, m), 3.07 (1 H, dd, J=12.8, 8.2 Hz), 2.62-2.74 (1 H, m),2.23-2.36(1 H, m), 1.13 (3 H, d, J=6.8 Hz) m/z 468.2(MH⁺). (4E)-3-methyl-N-pyridin-3-yl-4-(3-{{5-(trifluoromethyl)pyridin-2-yl]oxy]benzylidene)piperidine-1-carboxamide (enantiomer 2). Third eluting peak, t_{R}=2.60 min (4.6×150 mm Chiralpak AS-H, 20% ethanol/CO₂ at 3 mL/min).

### EXAMPLES 1-3

A solution of (4E)-3-methyl-N-pyridin-3-yl-4-(3-{[5-(trifluoromethyl)pyridin-2-yl]oxy}benzylidene)piperidine-1-carboxamide (1.00 g; enantiomer 1/second eluting peak) in 50% aqueous methanol was sealed in a vial and heated at 95° C. for 4 days. The solution was concentrated to dryness to give crude 2-{3-[(E)-(3-methylpiperidin-4-ylidene)methyl]phenoxy}-5-(trifluoromethyl)pyridine (902 mg) which as dissolved in acetonitrile to make a 0.5 M solution. In separate vials, 0.2 mL of the 0.5 M solution of 2-{3-[(E)-(3-methylpiperidin-4-ylidene)methyl]phenoxy}-5-(trifluoromethyl)pyridine (0.086 mmol, 1 equiv), triethylamine (0.10 mL, 7 equiv), and the appropriate phenyl heteroarylcarbamate (1.5 equiv) were combined and diluted with 0.50 mL of acetonitrile. After 30 min, the reactions were concentrated to dryness. The residues were dissolved in 1 mL DMSO and purified by reverse phase HPLC (acetonitrile/water/0.1% formic acid) to give Examples 1-3.

| | | | m/z |
|---|---|---|---|
| Ex. | Name | ¹H NMR (DMSO-d₆) δ ppm | (MH+) |
| 1 | (4E)-N-(3,4- | 1H NMR (400 MHz, DMSO-d6) δ ppm 8.58 (1 | 486.9 |
| | dimethylisoxazol-5-yl)-3- | H, br. s.), 8.24 (1 H, dd, J = 8.7, 2.4 Hz), 7.25 | |
| | methyl-4-(3-{[5- | (1 H, d, J = 8.5 Hz), 7.15 (2 H, d, J = 7.2 Hz), | |
| | (trifluoromethyl)pyridin-2- | 7.03-7.11 (2 H, m), 3.74 (1 H, dd, J = 12.9, | |
| | yl]oxy}benzylidene)piperi- | 4.0 Hz), 3.54-3.64 (1 H, m), 3.28-3.38 (1 H, | |
| | dine-1-carboxamide | m), 3.11 (1 H, dd, J = 12.8, 7.9 Hz), 2.61-2.71 | |
| | | (1 H, m), 2.42-2.49 (1 H, m), 2.25-2.35 (1 | |
| | | H, m), 2.13 (3 H, s), 1.75 (3 H, s), 1.14 (3 H, | |
| | | d, J = 6.7 Hz) | |
| 2 | (4E)-3-methyl-N-(5- | 1H NMR (400 MHz, DMSO-d₆) δ ppm 8.51 (1 | 473.1 |
| | methylisoxazol-4-yl)-4-(3-{[5- | H, d, J = 43.6 Hz), 8.21 (1 H, dd, J = 8.7, 2.3 | |
| | (trifluoromethyl)pyridin-2- | Hz), 8.10 (1 H, s), 7.42 (1 H, t, J = 7.8 Hz), 7.22 | |
| | yl]oxy}benzylidene)piperi- | (1 H, d, J = 8.6 Hz), 7.13 (1 H, d, J = 7.7 Hz), | |
| | dine-1-carboxamide | 7.00-7.08 (2 H, m), 6.36 (1 H, s), 3.74 (1 H, | |
| | | dd, J = 12.9, 3.9 Hz), 3.54-3.65 (1 H, m), 3.05 | |
| | | (1 H, dd, J = 12.8, 8.1 Hz), 2.64 (1 H, dd, | |
| | | J = 14.5, 4.6 Hz), 2.53 (2 H, s), 2.30 (4 H, s), | |
| | | 1.13 (3 H, d, J = 6.8 Hz) | |
| 3 | (4E)-N-isoxazol-4-yl-3- | 1H NMR (400 MHz, DMSO-d₆) δ ppm 8.82 (1 | 459.164 |
| | methyl-4-(3-{[5- | H, s), 8.51-8.59 (1 H, m), 8.21 (1 H, dd, | |
| | (trifluoromethyl)pyridin-2- | J = 8.8, 2.4 Hz), 7.42 (1 H, t, J = 7.8 Hz), 7.22 (1 | |
| | yl]oxy}benzylidene)piperi- | H, d, J = 8.8 Hz), 7.13 (1 H, d, J = 7.7 Hz), 7.00- | |
| | dine-1-carboxamide | 7.08 (2 H, m), 6.36 (1 H, s), 3.73 (1 H, dd, | |
| | | J = 12.9, 3.9 Hz), 3.55-3.65 (1 H, m), 3.06 (1 | |
| | | H, dd, J = 12.9, 8.1 Hz), 2.61-2.70 (1 H, m), | |
| | | 2.53 (3 H, s), 2.24-2.34 (1 H, m), 1.12 (3 H, | |
| | | d, J = 6.8 Hz) | |

The following compounds may be prepared by methods described herein and others known in the art;
(4E)-N-(3,4-dimethylisoxazol-5-yl)-3-methyl-4-(3-{[5-(trifluoromethyppyridin-2-yl]oxy}benzylidene)piperidine-1-carboxamide;
(4E)-3-methyl-N-(5-methylisoxazol-4-yl)-4-(3-{[5-(trifluoromethyl)pyridin-2-yl]oxy}benzylidene)piperidine-1-carboxamide;
(4E)-N-isoxazol-4-yl-3-methyl-4-(3-{[5-(trifluoromethyl)pyridin-2-yl]oxy}benzylidene)piperidine-1-carboxamide;
(4E)-4-{3-[(5-bromopyridin-2-yl)oxy]benzylidene}-N-(3,4-dimethylisoxazol-5-yl)-3-methylpiperidine-1-carboxamide;
(4E)-4-{3-[(5-bromopyridin-2-yl)oxy]benzylidene}-3-methyl-N-(5-methylisoxazol-4-yl)piperidine-1-carboxamide;
(4E)-4-{3-[(5-bromopyridin-2-yl)oxy]benzylidene}-N-isoxazol-4-yl-3-methylpiperidine-1-carboxamide;
(4E)-4-{3-[(5-bromopyridin-2-yl)oxy]benzylidene}-N-(4,5-dimethylisoxazol-3-yl)-3-methylpiperidine-1-carboxamide;
(4E)-4-{3-[(5-cyclopropylpyrimidin-2-yl)oxy]benzylidene}-N-(3,4-dimethylisoxazol-5-yl)-3-methylpiperidine-1-carboxamide;
(4E)-4-{3-[(5-cyclopropylpyrimidin-2-yl)oxy]benzylidene}-3-methyl-N-(5-methylisoxazol-4-yl)piperidine-1-carboxamide;
(4E)-4-{3-[(5-cyclopropylpyrimidin-2-yl)oxy]benzylidene}-N-isoxazol-4-yl-3-methylpiperidine-1-carboxamide;
(4E)-4-{3-[(5-cyclopropylpyrimidin-2-yl)oxy]benzylidene}-N-(4,5-dimethylisoxazol-3-yl)-3-methylpiperidine-1-carboxamide;
(4E)-N-(3,4-dimethylisoxazol-5-yl)-3-methyl-4-{3-[(5-methylpyrimidin-2-yl)oxy]benzylidene}piperidine-1-carboxamide;
(4E)-3-methyl-N-(5-methylisoxazol-4-yl)-4-{3-[(5-methylpyrimidin-2-yl)oxy]benzylidene}piperidine-1-carboxamide;
(4E)-N-isoxazol-4-yl-3-methyl-4-{3-[(5-methylpyrimidin-2-yl)oxy]benzylidene}piperidine-1-carboxamide;
(4E)-N-(4,5-dimethylisoxazol-3-yl)-3-methyl-4-{3-[(5-methylpyrimidin-2-yl)oxy]benzylidene}piperidine-1-carboxamide;
(4E)-N-(3,4-dimethylisoxazol-5-yl)-3-methyl-4-{3-[(5-methylpyridin-2-yl)oxy]benzylidene}piperidine-1-carboxamide;
(4E)-3-methyl-N-(5-methylisoxazol-4-yl)-4-{3-[(5-methylpyridin-2-yl)oxy]benzylidene}piperidine-1-carboxamide;
(4E)-N-isoxazol-4-yl-3-methyl-4-{3-[(5-methylpyridin-2-yl)oxy]benzylidene}piperidine-1-carboxamide;
(4E)-N-(4,5-dimethylisoxazol-3-yl)-3-methyl-4-{3-[(5-methylpyridin-2-yl)oxy]benzylidene}piperidine-1-carboxamide;
(4E)-N-(4,5-dimethylisoxazol-3-yl)-3-methyl-4-(3-{[5-(trifluoromethyl)pyridin-2-yl]oxy}benzylidene)piperidine-1-carboxamide;
(4E)-N-(3,4-dimethylisoxazol-5-yl)-3-methyl-4-(3-{[6-(trifluoromethyl)pyridin-3-yl]oxy}benzylidene)piperidine-1-carboxamide;
(4E)-3-methyl-N-(5-methylisoxazol-4-yl)-4-(3-{[6-(trifluoromethyl)pyridin-3-yl]oxy}benzylidene)piperidine-1-carboxamide;
(4E)-N-isoxazol-4-yl-3-methyl-4-(3-{[6-(trifluoromethyl)pyridin-3-yl]oxy}benzylidene)piperidine-1-carboxamide;
(4E)-N-(4,5-dimethylisoxazol-3-yl)-3-methyl-4-(3-{[6-(trifluoromethyl)pyridin-3-yl]oxy}benzylidene)piperidine-1-carboxamide;
(4E)-4-{3-[(5-bromopyrimidin-2-yl)oxy]benzylidene}-N-(3,4-dimethylisoxazol-5-yl)-3-methylpiperidine-1-carboxamide;
(4E)-4-{3-[(5-bromopyrimidin-2-yl)oxy]benzylidene}-3-methyl-N-(5-methylisoxazol-4-yl)piperdine-1-carboxamide;
(4E)-4-{3-[(5-bromopyrimidin-2-yl)oxy]benzylidene}-N-isoxazol-4-yl-3-methylpiperidine-1-carboxamide;
(4E)-4-{3-[(5-bromopyrimidin-2-yl)oxy]benzylidene}-N-(4,5-dimethylisoxazol-3-yl)-3-methylpiperidine-1-carboxamide;
(4E)-4-{3-[(5-chloropyridin-2-yl)oxy]benzylidene}-N-(3,4-dimethylisoxazol-5-yl)-3-methylpiperidine-1-carboxamide;
(4E)-4-{3-[(5-chloropyridin-2-yl)oxy]benzylidene}-3-methyl-N-(5-methylisoxazol-4-yl)piperidine-1-carboxamide;
(4E)-4-{3-[(5-chloropyridin-2-yl)oxy]benzylidene}-N-isoxazol-4-yl-3-methylpiperidine-1-carboxamide;
(4E)-4-{3-[(5-chloropyridin-2-yl)oxy]benzylidene}-N-(4,5-dimethylisoxazol-3-yl)-3-methylpiperidine-1-carboxamide; (
4E)-4-{3-[(5-cyclopropylpyridin-2-yl)oxy]benzylidene}-N-(3,4-dimethylisoxazol-5-yl-3-methylpiperidine-1-carboxamide;
(4E)-4-{3-[(5-cyclopropylpyridin-2-yl)oxy]benzylidene}-3-methyl-N-(5-methylisoxazol-4-yl)piperidine-1-carboxamide;
(4E)-4-{3-[(5-cyclopropylpyridin-2-yl)oxy]benzylidene}-N-isoxazol-4-yl-3-methylpiperidine-1-carboxamide;
(4E)-4-{3-[(5-cyclopropylpyridin-2-yl)oxy]benzylidene}-N-(4,5-dimethylisoxazol-3-yl)-3-methylpiperidine-1-carboxamide;
(4E)-N-(3,4-dimethylisoxazol-5-yl)-3-methyl-4-(3-{[5-(trifluoromethyl)pyrimidin-2-yl]oxy}benzylidene)piperidine-1-carboxamide;
(4E)-3-methyl-N-(5-methylisoxazol-4-yl)-4-(3-{[5-(trifluoromethyl)pyrimidin-2-yl]oxy}benzylidene)piperidine 1-carboxamide;
(4E)-N-isoxazol-4-yl-3-methyl-4-(3-{[5-(trifluoromethyl)pyrimidin-2-yl]oxy}benzylidene)piperidine-1-carboxamide;
(4E)-N-(4,5-dimethylisoxazol-3-yl)-3-methyl-4-(3-{[5-(trifluoromethyl)pyrimidin-2-yl]oxy}benzylidene)piperidine-1-carboxamide;
(4E)-4-{3-[(5-chloropyrimidin-2-yl)oxy]benzylidene}-N-(3,4-dimethylisoxazol-5-yl)-3-methylpiperidine-1-carboxamide;
(4E)-4-{3-[(5-chloropyrimidin-2-yl)oxy]benzylidene}-3-methyl-N-(5-methylisoxazol-4-yl)piperidine-1-carboxamide;
(4E)-4-{3-[(5-chloropyrimidin-2-yl)oxy]benzylidene}-N-isoxazol-4-yl-3-methylpiperidine-1-carboxamide;
(4E)-4-{3-[(5-chloropyrimidin-2-yl)oxy]benzylidene}-N-(4,5-dimethylisoxazol-3-yl)-3-methylpiperidine-1-carboxamide;
(4E)-N-(3,4-dimethylisoxazol-5-yl)-3-methyl-4-{3-[(6-methylpyridin-3-yl)oxy]benzylidene}piperidine-1-carboxamide
(4E)-3-methyl-N-(5-methylisoxazol-4-yl)-4-{3-[(6-methylpyridin-3-yl)oxy]benzylidene}piperidine-1-carboxamide;
(4E)-N-isoxazol-4-yl-3-methyl-4-{3-[(6-methylpyridin-3-yl)oxy]benzylidene}piperidine-1-carboxamide
(4E)-N-(4,5-dimethylisoxazol-3-yl)-3-methyl-4-{3-[(6-methylpyridin-3-yl)oxy]benzylidene}piperidine-1-carboxamide;
(4E)-N-(3,4-dimethylisoxazol-5-yl)-3-methyl-4-{3-[(6-cyclopropylpyridin-3-yl)oxy]benzylidene}piperidine-1-carboxamide;
(4E)-3-methyl-N-(5-methylisoxazol-4-yl)-4-{3-[(6-cyclopropylpyridin-3-yl)oxy]benzylidene}piperidine-1-carboxamide;
(4E)-N-isoxazol-4-yl-3-methyl-4-{3-[(6-cyclopropylpyridin-3-yl)oxy]benzylidene}piperidine-1-carboxamide; and
(4E)-N-(4,5-dimethylisoxazol-3-yl)-3-methyl-4-{3-[(6-cyclopropylpyridin-3-yl)oxy]benzylidene}piperidine-1-carboxamide; or a pharmaceutically acceptable salt thereof.

The biological activities of compounds described in the above examples were determined using the following assay.

FAAH Assay

A FAAH enzyme assay may be carried out as described in W02006/085196 in 384-well clear polystyrene plates in a total volume of 50 μl per well. All percents are by volume. To each well was placed the reaction mixture (40 μl) containing 1-4 nM FAAH, 50 mM NaPᵢ, pH 7.4, 3 mM α-ketoglutarate, 0.15 mM NADH, 7.5 U/ml glutamate dehydrogenase, 2 mM ADP, 1 mM EDTA, and 0.1% Triton X-100 (The concentration shown for each component is the final concentration in the assay). To this mixture was added 5 μl of a compound of Examples 1 to 20 at various concentrations prepared in 50% DMSO (or 5 μl 50% DMSO for controls). This was immediately followed by the addition of 5 μl oleamide (500 μM) dissolved in 75% EtOH/25% DMSO and the reaction mixture is mixed for 1.5 min. The final concentrations of DMSO and EtOH in the assay were each 7.5%. The reactions was then incubated at 30° C. and the absorbance at 340 nm was collected over a period of 90 min with readings taken in 30-second intervals using SpectraMax Plus³⁸⁴ Microplate Spectrophotometer (Molecular Devices, Sunnyvale, Calif.). Human FAAH used in the assay was prepared as described in the patent application WO 2006/067613. The purity of the enzyme used was greater than 98% based on an analysis by SDS-polyacrylamide gel electrophoresis followed by Coomassie Blue staining.

Kinetic Data Analyses

Initial velocity data (V) is obtained from the slopes of the initial progressive curves. They are plotted as a function of substrate concentration and fit to the Michaelis-Menten equation (1) using Prism (GraphPad Software, Inc., San Diego, Calif.) software to obtain Kₘ and Vₘₐₓ values.

$$V = \frac{{V}_{max} [ S ]}{{K}_{m} + [ S ]} \qquad ( 1 )$$

To obtain potencies of irreversible inhibitors, progressive curves consistent with first order inhibition kinetics (two-step irreversible inhibition mechanism) are fit to equation (2) by nonlinear least squares regressions to determine k_{obs} values at each inhibitor concentration, where [P]ₜ is the absorbance at time t, V₀ is a constant related to the steady state rate of the uninhibited reaction, and k_{obs} is the first order rate constant for enzyme inactivation. The inhibitor dissociation constant (Kᵢ) and the first order rate

$${[ P ]}_{t} = {V}_{o} \frac{( 1 - {e}^{- {k}_{obs} t} )}{{k}_{obs}} \qquad ( 2 )$$

constant of enzyme inactivation at infinite inhibitor concentration (k_{inact}) are then obtained by fitting the k_{obs} vs. [I] curves to equation (3).

$${k}_{obs} = \frac{{k}_{inact} [ I ]}{[ I ] + {K}_{i} ( 1 + \frac{[ S ]}{{K}_{m}} )} \qquad ( 3 )$$

| | hFAAH |
|---|---|
| | K_{inact}/Kᵢ |
| Ex. | (M⁻¹s⁻¹) |
| 1 | 624 |
| 2 | 2850 |
| 3 | 4350 |

1. A compound of Formula I 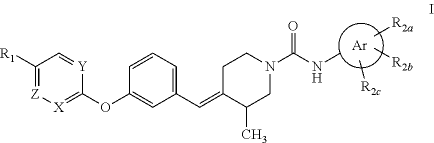
wherein:
Ar is a 5-membered heteroaryl moiety;
X, Y and Z are independently selected from CH or N;
R₁ is H, C₁-C₆ alkyl, C₁-C₆ alkoxy, halogen, C₁-C₃ haloalkyl, C₁-C₃ haloalkoxy, C₃-C₆ cycloalkyl, —CH₂—C₃-C₆ cycloalkyl, C₃-C₆ cycloalkoxy, —CH₂—C₃-C₆ cycloalkoxy, or CN; with the rings of the C₃-C₆ cycloalkyl, —CH₂—C₃-C₆ cycloalkyl, C₃-C₆ cycloalkoxy, and —CH₂—C₃-C₆ cycloalkoxy groups being optionally substituted by from 1 to 4 fluorine atoms;
R₂ₐ is H, C₁-C₆ alkyl, C₁-C₆ alkoxy, C₂-C₆ alkenyl, C₂-C₆ alkynyl, halogen, C₁-C₃ haloalkyl, C₁-C₃ haloalkoxy, C₃-C₈ cycloalkyl, —(CH₂)ₙ—C₃-C₈ cycloalkyl, C₃-C₈ cycloalkoxy, C₅-C₈ cycloalkenyl, —(CH₂)ₙ—C₅-C₈ cycloalkenyl,C₅-C₈ cycloalkenyloxy, 4- to 8-membered heterocycle containing from 1 to 3 ring heteroatoms selected from O, S and N, —(CH₂)ₙ-(4- to 8-membered heterocycle containing from 1 to 3 ring heteroatoms selected from O, S and N), —(CH₂)ₙ—O-(4- to 8-membered heterocycle containing from 1 to 3 ring heteroatoms selected from O, S and N) or CN; with:
a) the R₂ₐ C₃-C₈ cycloalkyl, —(CH₂)ₙ—C₃-C₈ cycloalkyl, C₃-C₈ cycloalkoxy, C₅-C₈ cycloalkenyl, —(CH₂)ₙ—C₅-C₈ cycloalkenyl, C₅-C₈ cycloalkenyloxy, 4- to 8-membered heterocycle containing from 1 to 3 ring heteroatoms selected from O, S and N, —(CH₂)ₙ-(4- to 8-membered heterocycle containing from 1 to 3 ring heteroatoms selected from O, S and N) and —(CH₂)ₙ—O-(4- to 8-membered heterocycle containing from 1 to 3 ring heteroatoms selected from O, S and N) groups being further optionally substituted by from 1 to 4 groups selected from halo, CN, —CH₂—CN, —CH₃, —CH₂F, —CHF₂, CF₃, —O—CH₃, —O—CH₂F, -O-CHF₂, or —O—CF₃;
b) the —(CH₂)ₙ- linking group of the —(CH₂)ₙ-C₃-C₈ cycloalkyl, —(CH₂)ₙ—C₅-C₈ cycloalkenyl, and —(CH₂)ₙ-(4- to 8-membered heterocycle containing from 1 to 3 ring heteroatoms selected from O, S and N) and —(CH₂)ₙ—O-(4- to 8-membered heterocycle containing from 1 to 3 ring heteroatoms selected from O, S and N) groups being further optionally substituted by from 1 to 4 groups selected from halo, CN, —CH₂—CN, —CH₃, —CH₂F, —CHF₂, CF₃, —O—CH₃, —O—CH₂F, —O—CHF₂, or —O—CF₃;
with R₂ₐ also optionally being a phenyl or pyridyl group optionally substituted by from 1 to 3 substituents selected from H, CN, —CH₂—CN, halogen, C₁-C₃ alkyl, —CH₂F, —CHF₂, CF₃, —O—C₁-C₃ alkyl, —O—CH₂F, —O—CHF₂, or —O—CF₃; and
R_{2b} and R_{2c} are independently selected from H, halogen, CN, —CH₂—CN, C₁-C₃ alkyl, C₁-C₃ alkoxy, —CH₂F, —CHF₂, CF₃, —O—CH₂F, —O—CHF₂, or —O—CF₃;
or a pharmaceutically acceptable salt thereof.

2. A compound of formula I selected from:
(4E)-N-(3,4-dimethylisoxazol-5-yl)-3-methyl-4-(3-{[5-(trifluoromethyl)pyridin-2-yl]oxy}benzylidene)piperidine-1-carboxamide;
(4E)-3-methyl-N-(5-methylisoxazol-4-yl)-4-(3-{[5-(trifluoromethyl)pyridin-2-yl]oxy}benzylidene)piperidine-1-carboxamide;
(4E)-N-isoxazol-4-yl-3-methyl-4-(3-{[5-(trifluoromethyl)pyridin-2-yl]oxy}benzylidene)piperidine-1carboxamide;
(4E)-N-(3,4-dimethylisoxazol-5-yl)-3-methyl-4-(3-{[5-(trifluoromethyl)pyridin-2-yl]oxy}benzylidene)piperidine-1-carboxamide;
(4E)-3-methyl-N-(5-methylisoxazol-4-yl)-4-(3-{[5-(trifluoromethyl)pyridin-2-yl]oxy}benzylidene)piperidine-1 -carboxamide;
(4E)-N-isoxazol-4-yl-3-methyl-4-(3-{[5-(trifluoromethyl)pyridin-2-yl]oxy}benzylidene)piperidine-1-carboxamide;
(4E)-4-{3-[(5-bromopyridin-2-yl)oxy]benzylidene}-N-(3,4-dimethylisoxazol-5-yl)-3-methylpiperidine-1-carboxamide;
(4E)-4-{3-[(5-bromopyridin-2-yl)oxy]benzylidene}-3-methyl-N-(5-methylisoxazol-4-yl)piperidine-1-carboxamide;
(4E)-4-{3-[(5-bromopyridin-2-yl)oxy]benzylidene}-N-isoxazol-4-yl-3-methylpiperidine-1-carboxamide;
(4E)-4-{3-[(5-bromopyridin-2-y)oxy]benzylidene}-N-(4,5-dimethylisoxazol-3-yl)-3-methylpiperidine-1-carboxamide;
(4E)-4-{3-[(5-cyclopropylpyrimidin-2-yl)oxy]benzylidene}-N-(3,4-dimethylisoxazol-5-yl)-3-methylpiperidine-1-carboxamide;
(4E)-4-{3-[(5-cyclopropylpyrimidin-2-yl)oxy]benzylidene}-3-methyl-N-(5-methylisoxazol-4-yl)piperidine-1-carboxamide;
(4E)-4-{3-[(5-cyclopropylpyrimidin-2-yl)oxy]benzylidene}-N-isoxazol-4-yl-3-methylpiperidine-1-carboxamide;
(4E)-4-{3[(5-cyclopropylpyrimidin-2-yl)oxy]benzylidene}-N-(4,5-dimethylisoxazol-3-yl)-3-methylpiperidine-1-carboxamide;
(4E)-N-(3,4-d imethylisoxazol-5-yl)-3-methyl-4-{3-[(5-methylpyrimidin-2-yl)oxy]benzylidene}piperidine-1-carboxamide;
(4E)-3-methyl-N-(5-methylisoxazol-4-yl)-4-{3-[(5-methylpyrimidin-2-yl)oxy]benzylidene}piperidine-1-carboxamide;
(4E)-N-isoxazol-4-yl-3-methyl-4-{3-[(5-methylpyrimidin-2-yl)oxy]benzylidene}piperidine-1carboxamide;
(4E)-N-(4,5-dimethylisoxazol-3-yl)-3-methyl-4-{3-[(5-methylpyrimidin-2-yl)oxy]benzylidene}piperidine-1-carboxamide;
(4E)-N-(3,4-dimethylisoxazol-5-yl)-3-methyl-4-{3-[(5-methylpyridin-2-yl)oxy]benzylidene}piperidine-1-carboxamide;
(4E)-3-methyl-N-(5-methylisoxazol-4-yl)-4-{3-[(5-methylpyridin-2-yl)oxy]benzylidene}piperidine-1carboxamide;
(4E)-N-isoxazol-4-yl-3-methyl-4-(3-[(5-methylpyridin-2-yl)oxy]benzylidene}piperidine-1-carboxamide;
(4E)-N-(4,5-dimethylisoxazol-3-yl)-3-methyl-4-{3-[(5-methylpyridin-2-yl)oxy]benzylidene}piperidine-1-carboxamide;
(4E)-N-(4,5-dimethylisoxazol-3-yl)-3-methyl-4-(3-{[5-(trifluoromethyl)pyridin-2-yl]oxy}benzylidene)piperidine-1-carboxamide;
(4E)-N-(3,4-dimethylisoxazol-5-yl)-3-methyl-4-(3-{[6-(trifluoromethyl)pyridin-3-yl]oxy}benzylidene)piperidine-1-carboxamide;
(4E)-3-methyl-N-(5-methylisoxazol-4-yl)-4-(3-{[6-(trifluoromethyl)pyridin-3-yl]oxy}benzylidene)piperidine-1-carboxamide;
(4E)-N-isoxazol-4-yl-3-methyl-4-(3-{[6-(trifluoromethyl)pyridin-3-yl]oxy}benzylidene)piperidine-1-carboxamide;
(4E)-N-(4,5-dimethyl isoxazol-3-yl)-3-methyl-4-(3-{[6-(trifluoromethyl)pyridin-3-yl]oxy}benzylidene)piperidine-1-carboxamide;
(4E)-4-{3-[(5-bromopyrimidin-2-yl)oxy]benzylidene}-N-(3,4-dimethylisoxazol-5-yl)-3-methylpiperidine-1-carboxamide;
(4E)-4-{3-[(5-bromopyrimidin-2-yl)oxy]benzylidene}-3-methyl-N-(5-methylisoxazol-4yl)-piperidine-1-carboxamide;
(4E)-4-{3-[(5-bromopyrimidin-2-yl)oxy]benzylidene}-N-isoxazol-4-yl-3-methylpiperidine-1-carboxamide;
(4E)-4-{3-[(5-bromopyrimidin-2-yl)oxy]benzylidene}-N-(4,5-dimethylisoxazol-3-yl)-3-methylpiperidine-1-carboxamide;
(4E)-4-{3-[(5-chloropyridin-2-yl)oxy]benzylidene}-N-(3,4-dimethylisoxazol-5-yl)-3-methylpiperidine-1-carboxamide;
(4E)-4-{3-[(5-chloropyridin-2-yl)oxy]benzylidene}-3-methyl-N-(5-methylisoxazol-4-yl)piperidine-1-carboxamide;
(4E)-4-{3-[(5-chloropyridin-2-yl)oxy]benzylidene}-N-isoxazol-4-yl-3-methylpiperidine-1-carboxamide;
(4E)-4-{3-[(5-chloropyridin-2-yl)oxy]benzylidene}-N-(4,5-dimethylisoxazol-3-yl)-3-methylpiperidine-1-carboxamide;
(4E)-4-{3-[(5-cyclopropylpyridin-2-yl)oxy]benzylidene}-N-(3,4-dimethylisoxazol-5-yl)-3-methylpiperidine-1-carboxamide;
(4E)-4-{3-[(5-cyclopropylpyridin-2-yl)oxy]benzylidene}-3-methyl-N-(5-methylisoxazol-4-yl)piperidine-1-carboxamide;
(4E)-4-{3-[(5-cyclopropylpyridin-2-yl)oxy]benzylidene}-N-isoxazol-4-yl-3-methylpiperidine-1-carboxamide;
(4E)-4-{3-[(5-cyclopropylpyridin-2-yl)oxy]benzylidene}-N-(4,5-dimethylisoxazol-3-yl)-3-methylpiperidine-1-carboxamide;
(4E)-N-(3,4-dimethylisoxazol-5-yl)-3-methyl-4-(3-{[5-(trifluoromethyl)pyrimidin-2-yl]oxy}benzylidene)piperidine-1-carboxamide;
(4E)-3-methyl-N-(5-methylisoxazol-4-yl)-4-(3-{[5-(trifluoromethyl)pyrimidin-2-yl]oxy}benzylidene)piperidine-1-carboxamide;
(4E)-N-isoxazol-4-yl-3-methyl-4-(3-{[5-(trifluoromethyl)pyrimidin-2-yl]oxy}benzylidene)piperidine-1-carboxamide;
(4E)-N-(4,5-dimethylisoxazol-3-yl)-3-methyl-4-(3-{[5-(trifluoromethyl)pyrimidin-2-yl]oxy}benzylidene)piperidine-1-carboxamide;
(4E)-4-{3-[(5-chloropyrimidin-2-yl)oxy]benzylidene}-N-(3,4-dimethylisoxazol-5-yl)-3-methylpiperidine-1-carboxamide;
(4E)-4-{3-[(5-chloropyrimidin-2-yl)oxy]benzylidene}-3-methyl-N-(5-methylisoxazol-4-yl)piperidine-1-carboxamide;
(4E)-4-{3-[(5-chloropyrimidin-2-yl)oxy]benzylidene}-N-isoxazol-4-yl-3-methylpiperidine-1-carboxamide;
(4E)-4-{3-[(5-chloropyrimidin-2-yl)oxy]benzylidene}-N-(4,5-dimethylisoxazol-3-yl)-3-methylpiperidine-1-carboxamide;
(4E)-N-(3,4-dimethylisoxazol-5-yl)-3-methyl-4-{3-[(6-methylpyridin-3-yl)oxy]benzylidene}piperidine-1-carboxamide
(4E)-3-methyl-N-(5-methylisoxazol-4-yl)-4-{3-[(6-methylpyridin-3-yl)oxy]benzylidene}piperidine-1-carboxamide;
(4E)-N-isoxazol-4-yl-3-methyl-4-{3-[(6-methylpyridin-3-yl)oxy]benzylidene}piperidine-1-carboxamide
(4E)-N-(4,5-dimethylisoxazol-3-yl)-3-methyl-4-{3-[(6-methylpyridin-3-yl)oxy]benzylidene}piperidine-1-carboxamide;
(4E)-N-(3,4-dimethylisoxazol-5-yl)-3-methyl-4-{3-[(6-cyclopropylpyridin-3yl)oxy]benzylidene}piperidine-1-carboxamide;
(4E)-3-methyl-N-(5-methylisoxazol-4-yl)-4-{3[(6-cyclopropylpyridin-3-yl)oxy]benzylidene}piperidine-1-carboxamide;
(4E)-N-isoxazol-4-yl-3-methyl-4-{3-[(6-cyclopropylpyridin-3-yl)oxy]benzylidene}piperidine-1-carboxamide; and
(4E)-N-(4,5-dimethylisoxazol-3-yl)-3-methyl-4-{3-[(6-cyclopropylpyridin-3-yl)oxy]benzylidene}piperidine-1-carboxamide;
or a pharmaceutically acceptable salt thereof.

3. A pharmaceutical composition comprising a compound according to claim 1, or a pharmaceutically acceptable salt thereof, and a pharmaceutically acceptable carrier, and optionally a further therapeutic agent,

4-8. (canceled)

9. A method of treating a FAAH-mediated disease or condition comprisiing administration of a compound according to claim 1 or a pharmaceutically acceptable salt thereof.

10. A method according to claim 9 wherein the FAAH-mediated disease or condition is acute pain, chronic pain, neuropathic pain, nociceptive pain, inflammatory pain, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, or cardiovascular disease.
